(19) United States
(12) Patent Application Publication
(10) Pub. No.: US 2014/0128571 A1
(43) Pub. Date: May 8, 2014

(54) AROMATIC COMPOUNDS WITH SULFUR CONTAINING LIGANDS

(71) Applicant: THE UNIVERSITY OF KENTUCKY RESEARCH FOUNDATION, Lexington, KY (US)

(72) Inventors: Boyd E. Haley, Nicholasville, KY (US); Niladri Narayan Gupta, Georgetown, KY (US)

(73) Assignee: THE UNIVERSITY OF KENTUCKY RESEARCH FOUNDATION, Lexington, KY (US)

(21) Appl. No.: 14/147,990

(22) Filed: Jan. 6, 2014

Related U.S. Application Data
(62) Division of application No. 13/763,057, filed on Feb. 8, 2013.
Application No. 13/763,057 is a continuation-in-part of application No. 13/565,047, filed on Aug. 2, 2012, now abandoned.
Application No. 13/565,047 is a continuation of application No. 12/731,415, filed on Mar. 25, 2010, now abandoned.

(51) Int. Cl. C07D 213/81; C07K 5/083; C07D 213/82

(52) U.S. Cl. CPC C07D 213/81; C07D 213/82; C07K 5/0806
USPC 530/331; 546/257

(57) ABSTRACT

Compounds useful as nutritional supplements, antioxidants, heavy metal chelators and/or as intermediates for producing other related compounds with like uses have a formula:

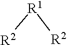

where R¹ is an aromatic backbone and R² is a sulfur containing ligand.

This application is a division of U.S. patent application Ser. No. 13/763,057 file Feb. 8, 2013, which is a continuation-in-part of U.S. patent application Ser. No. 13/565,047 filed 2 Aug. 2012, which is a continuation of U.S. patent application Ser. No. 12/731,415 filed on 25 Mar. 2010, the full disclosures are incorporated herein by reference.

### TECHNICAL FIELD

The present invention relates generally to novel aromatic compounds useful as nutritional supplements, antioxidants, heavy metal chelators and/or also as intermediates for producing other useful compounds of this type.

### BACKGROUND OF THE INVENTION

Free radicals are unstable oxygen-containing molecules that negatively interact with other molecules in the body, in a process called oxidation. High levels of free radicals and oxidation can lead to oxidative stress. Moderate oxidative stress can trigger apoptosis: a genetically determined process of cell self destruction marked by fragmentation of nuclear DNA. More intensive oxidative stress may cause widespread necrosis or cell death.

The body naturally fights oxidation by producing glutathione (GSH). Glutathione is a tripeptide composed of three amino acid residues: glutamic acid, cysteine and glycine. Glutathione is found in all cells in the body, including the bile, the epithial lining fluid of the lungs and in the blood. Glutathione is the smallest intracellular protein thiol molecule in the cells (that is: a molecule containing an —SH or sulfhydryl group). This characteristic emphasizes its potent antioxidant action and supports a multi-faceted thiol exchange system which regulates cell activity. Glutathione is responsible for three crucial protective functions. Without it, cells disintegrate from unrestrained oxidation, the body would have little resistance to metabolic acids and the liver would shrivel up from the eventual accumulation of acidic toxins.

As noted above, the body naturally fights oxidation by producing glutathione. Once glutathione stabilizes a free radical it becomes oxidized and is usually excreted from the body. Thus, the body must replace glutathione as it is used. It should be appreciated that high levels of oxidative stress can prevent the body from recovering its normal function. The present invention relates to novel compounds useful as antioxidant dietary supplements that help maintain a healthy glutathione level allowing the body to maintain its own natural detoxifying capacity even when subjected to high levels of oxidative stress over an extended period of time.

### SUMMARY OF THE INVENTION

In accordance with the objects and advantages of the present invention, novel aromatic compounds are provided incorporating sulfur containing ligands. The chemical compounds comprise:

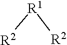

where R¹=

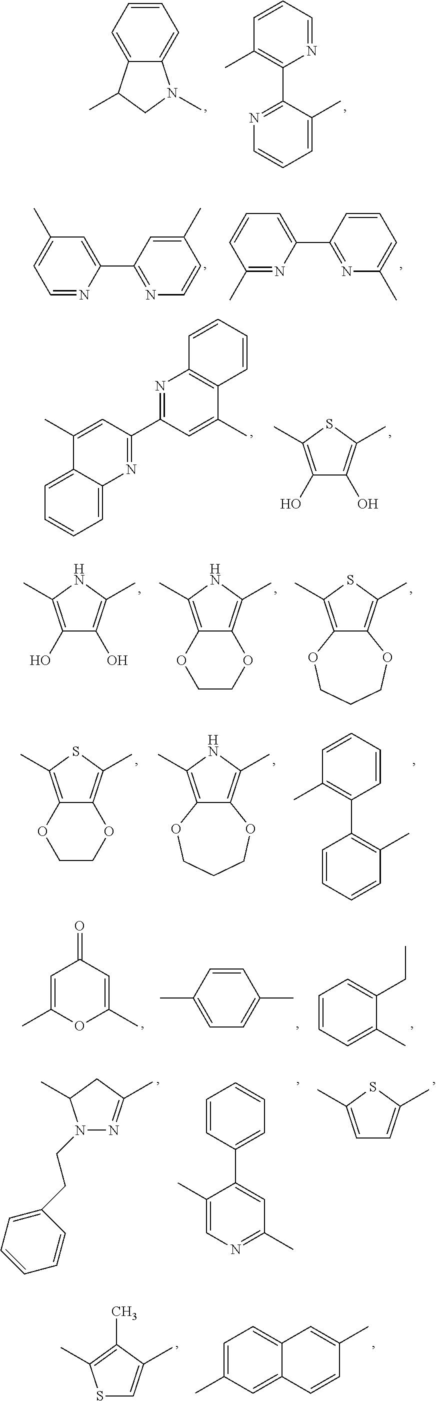

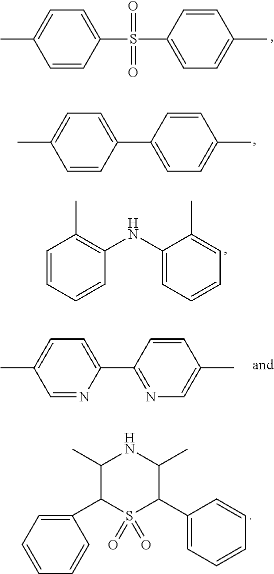

R²=

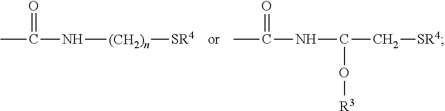

R³=ethyl or methyl, R⁴=hydrogen, glutathione, cysteine, alpha dihidrolipoic acid, aptamine, thiolphosphate, 5′thioladenosine, L-homocysteine, co-enzyme A, 2-mercaptoethanol, dithiothreitol, iodoacetate, bromoacetate, fluoroacetate or chloroacetate and n=2-4.

Other aspects of the present invention will become apparent to those skilled in this art from the following description wherein there is shown and described exemplary embodiments of this invention. As it will be realized, the invention is capable of further embodiments and its several details are capable of modification in various, obvious aspects all without departing from the invention. Accordingly, the drawings and descriptions will be regarded as illustrative in nature and not as restrictive.

### DETAILED DESCRIPTION OF THE INVENTION

As noted above, the present invention relates to novel aromatic compounds, incorporating sulfur containing ligands that are useful as nutritional supplements, antioxidants, heavy metal chelators and as intermediates for the synthesis of other related useful compounds. The chemical compounds of the present invention may be broadly described as comprising:

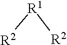

where R¹=

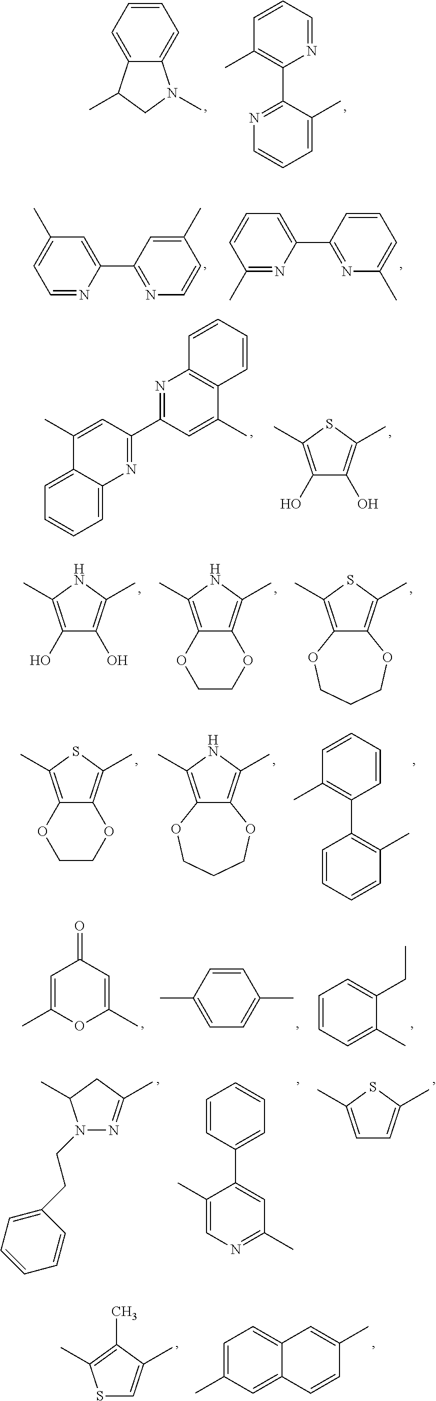

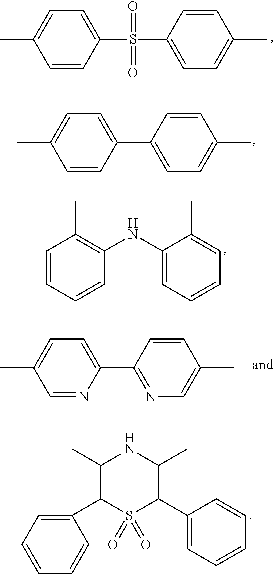

R²=

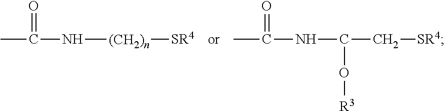

R³=ethyl or methyl, R⁴=hydrogen, glutathione, cysteine, alpha dihidrolipoic acid, aptamine, thiolphosphate, 5′thioladenosine, L-homocysteine, co-enzyme A, 2-mercaptoethanol, dithiothreitol, iodoacetate, bromoacetate, fluoroacetate or chloroacetate and n=2-4.

The compounds of the present invention exhibit a number of unique properties that make them attractive for use in methods of (a) supplementing a diet, (b) removing heavy metals and other toxins and (c) ameliorating oxidative stress in mammals. Generally the compounds exhibit low toxicity. The compounds are also generally lipid soluble and, accordingly, after entering the plasma the compounds can enter cells of all tissues, cross the blood/brain barrier and enter the bone marrow. This is important because the damage caused by heavy metals and the oxidative stress produced by hydroxyl free radicals and other free radicals of the reactive oxygen species mostly occur in the intercellular space. In contrast, most dietary antioxidants are water soluble and cannot enter into cells effectively nor can they cross the blood/brain barrier.

It is expected that pharmaceutical compositions may be prepared by combining a pharmaceutically effective amount of a compound of the present invention with an appropriate excipient. Such compositions should be effective via various types of administration including, but not limited to, oral administration, transdermal administration, nasal administration, administration by suppository, intravenous administration and the like.

The following synthesis and examples are presented to further illustrate the invention, but it is not to be considered as limited thereto. In the examples, S-Cys refers to cysteine having a structural formula: —SCH₂CH(NH₂)COOH GS refers to glutathione having a structural formula:

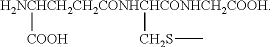

### Example 1

### Compounds 1-9

Starting Material: Indole-1,3-dicarboxylic acid diethyl ester. Aldrich Catalog number R 163023. M.W: 261.28

### Compound 1

Indole-1,3-dicarboxylic acid diethyl ester (2.61 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 1.

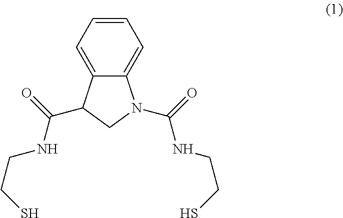 [SMILES: O=C(NCCS)C1CN(C(=O)NCCS)c2ccccc21]

### Compound 2

Compound 1 (0.325 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 2. The purity is checked with thin layered chromatography on PEI cellulose matrix developed with 0.4M ammonium bicarbonate solution.

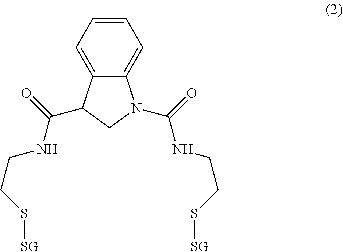

### Compound 3

Compound 1 (0.325 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 3. The purity is checked with thin layered chromatography on PEI cellulose matrix developed with 0.4M ammonium bicarbonate solution.

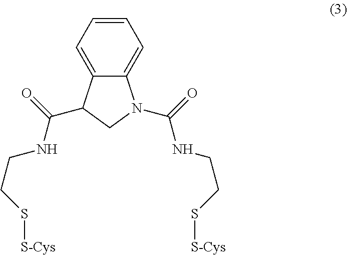

### Compound 4

Indole-1, 3 dicarboxylic acid diethyl ester (2.61 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 4.

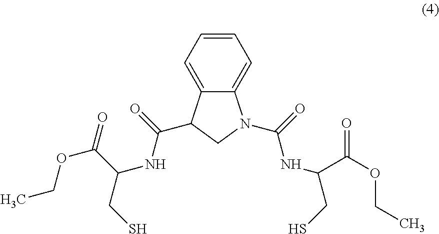 [SMILES: CCOC(=O)C(CS)NC(=O)C1CN(C(=O)NC(CS)C(=O)OCC)c2ccccc21]

### Compound 5

Compound 4 (0.469 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 5. The purity is checked with thin layered chromatography on PEI cellulose matrix developed with 0.4M ammonium bicarbonate solution.

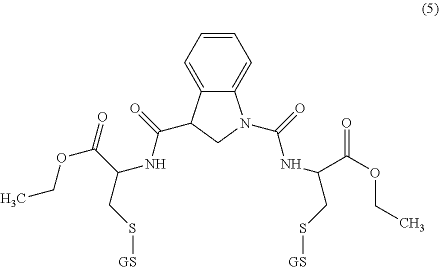

### Compound 6

Compound 4 (0.469 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 6. The purity is checked with thin layered chromatography on PEI cellulose matrix developed with 0.4M ammonium bicarbonate solution.

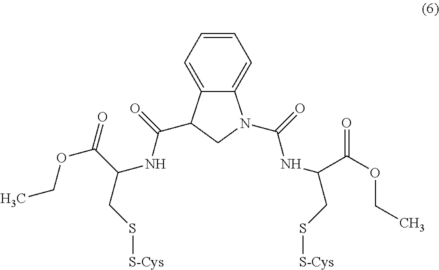

### Compound 7

Indole-1, 3 dicarboxylic acid diethyl ester (2.61 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 7

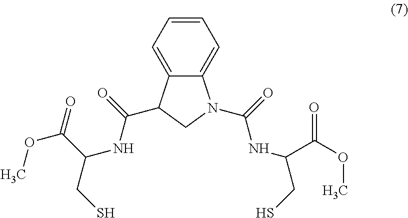

### Compound 8

Compound 7 (0.441 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 8. The purity is checked with thin layered chromatography on PEI cellulose matrix developed with 0.4M ammonium bicarbonate solution.

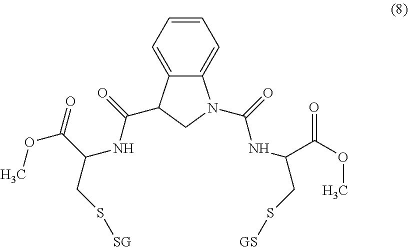

### Compound 9

Compound 7 (0.441 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 9. The purity is checked with thin layered chromatography on PEI cellulose matrix developed with 0.4M ammonium bicarbonate solution.

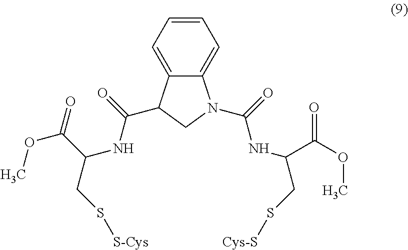

Compounds 10-18

Starting Material: 2,2′-bipyridine-3,3′-dicarboxylic acid. Aldrich Catalog number: 457191. M.W: 244.20

### Compound 10

2,2′-bipyridine-3,3′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 10

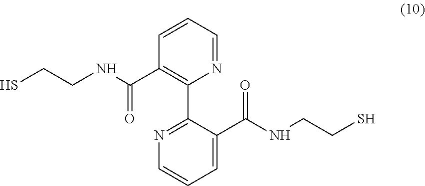 [SMILES: O=C(NCCS)c1cccnc1-c1ncccc1C(=O)NCCS]

### Compound 11

Compound 10 (0.362 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 11. The purity is checked with thin layered chromatography on PEI cellulose matrix developed with 0.4M ammonium bicarbonate solution.

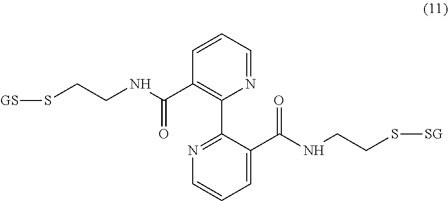

### Compound 12

Compound 10 (0.362 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 12. The purity is checked with thin layered chromatography on PEI cellulose matrix developed with 0.4M ammonium bicarbonate solution.

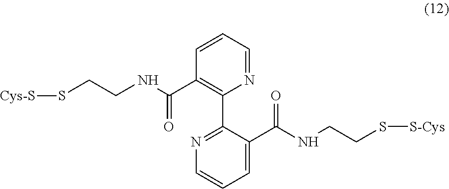

### Compound 13

2,2′-bipyridine-3,3′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 13

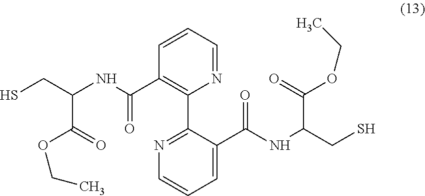 [SMILES: CCOC(=O)C(CS)NC(=O)c1cccnc1-c1ncccc1C(=O)NC(CS)C(=O)OCC]

### Compound 14

Compound 13 (0.506 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 14. The purity is checked with thin layered chromatography on PEI cellulose matrix developed with 0.4M ammonium bicarbonate solution.

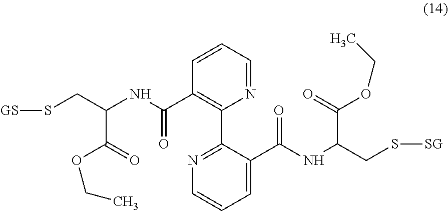

### Compound 15

Compound 13 (0.506 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 15. The purity is checked with thin layered chromatography on PEI cellulose matrix developed with 0.4M ammonium bicarbonate solution.

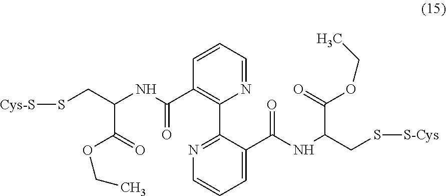

### Compound 16

2,2′bipyridine-3,3′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 16.

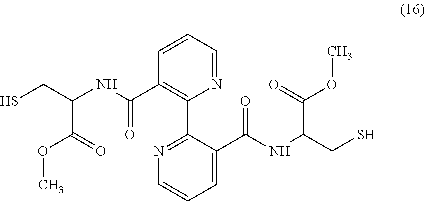 [SMILES: COC(=O)C(CS)NC(=O)c1cccnc1-c1ncccc1C(=O)NC(CS)C(=O)OC]

### Compound 17

Compound 16 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 17. The purity is checked with thin layered chromatography on PEI cellulose matrix developed with 0.4M ammonium bicarbonate solution.

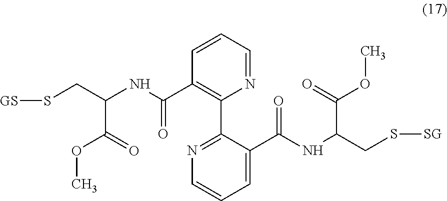

### Compound 18

Compound 16 (0.478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 18. The purity is checked with thin layered chromatography on PEI cellulose matrix developed with 0.4M ammonium bicarbonate solution.

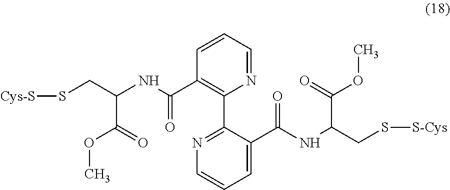

Compounds 19-27

Starting Material: 2,2′-bipyridine-4,4′-dicarboxylic acid. Aldrich Catalog number: 457191. M.W: 244.20

### Compound 19

2,2′-bipyridine-4,4′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 19.

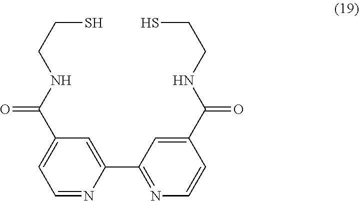 [SMILES: O=C(NCCS)c1ccnc(-c2cc(C(=O)NCCS)ccn2)c1]

### Compound 20

Compound 19 (0.362 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 20. The purity is checked with thin layered chromatography on PEI cellulose matrix developed with 0.4M ammonium bicarbonate solution.

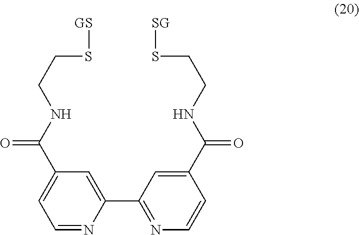

### Compound 21

Compound 19 (0.362 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 21. The purity is checked with thin layered chromatography on PEI cellulose matrix developed with 0.4M ammonium bicarbonate solution.

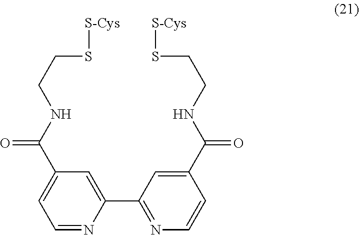

### Compound 22

2,2′-bipyridine-4,4′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 22.

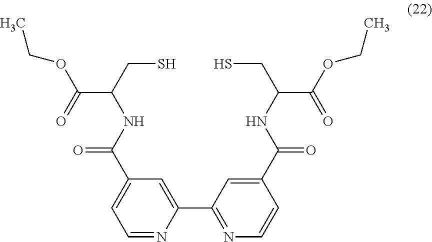 [SMILES: CCOC(=O)C(CS)NC(=O)c1ccnc(-c2cc(C(=O)NC(CS)C(=O)OCC)ccn2)c1]

### Compound 23

Compound 22 (0.506 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 23. The purity is checked with thin layered chromatography on PEI cellulose matrix developed with 0.4M ammonium bicarbonate solution.

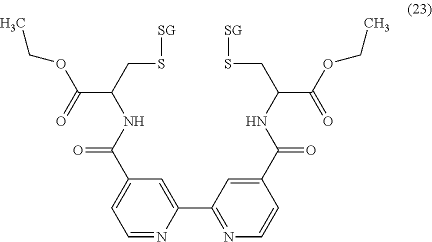

### Compound 24

Compound 22 (0.506 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 24. The purity is checked with thin layered chromatography on PEI cellulose matrix developed with 0.4M ammonium bicarbonate solution.

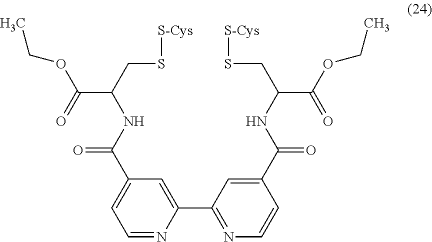

### Compound 25

2,2′bipyridine-4,4′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 25.

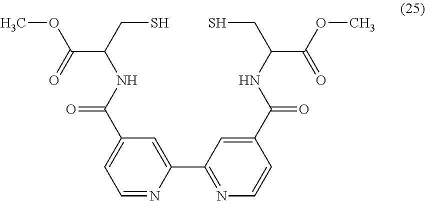

### Compound 26

Compound 25 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 26. The purity is checked with thin layered chromatography on PEI cellulose matrix developed with 0.4M ammonium bicarbonate solution.

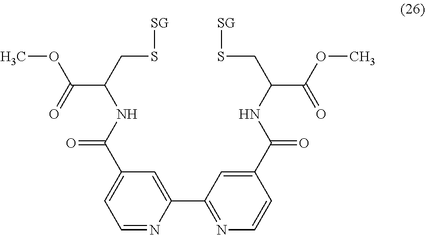

### Compound 27

Compound 25 (0.478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 27. The purity is checked with thin layered chromatography on PEI cellulose matrix developed with 0.4M ammonium bicarbonate solution.

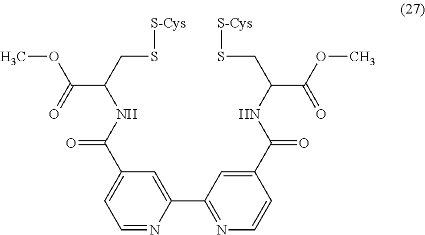

Compounds 28-36

Starting Material: 2,2′-bipyridine-6,6′-dicarboxylic acid J&K Scientific, Ltd. Product No. 28775 M.W: 244.20

### Compound 28

2,2′-bipyridine-6,6′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 28.

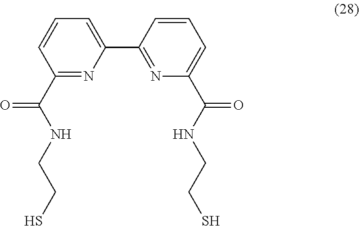 [SMILES: O=C(NCCS)c1cccc(-c2cccc(C(=O)NCCS)n2)n1]

### Compound 29

Compound 28 (0.362 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 29. The purity is checked with thin layered chromatography on PEI cellulose matrix developed with 0.4M ammonium bicarbonate solution.

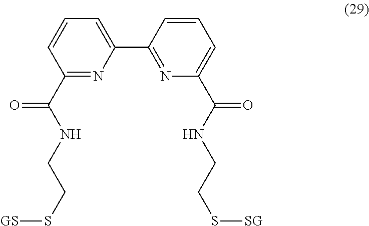

### Compound 30

Compound 28 (0.362 gm, 1 mmol) is dissolved in 10 ml of Dimethyl formamide and 5 ml of water is added to the mixture. L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 30. The purity is checked with thin layered chromatography on PEI cellulose matrix developed with 0.4M ammonium bicarbonate solution.

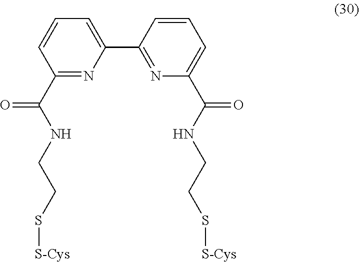

### Compound 31

2,2′-bipyridine-6,6′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 31.

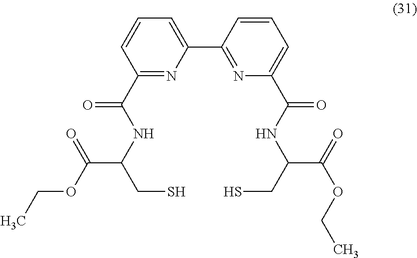 [SMILES: CCOC(=O)C(CS)NC(=O)c1cccc(-c2cccc(C(=O)NC(CS)C(=O)OCC)n2)n1]

### Compound 32

Compound 31 (0.506 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 32. The purity is checked with thin layered chromatography on PEI cellulose matrix developed with 0.4M ammonium bicarbonate solution.

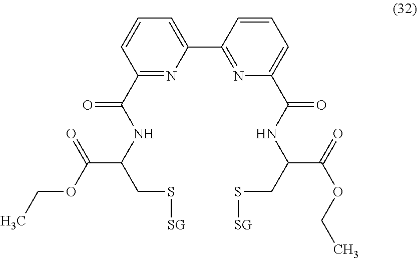

### Compound 33

Compound 31 (0.506 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 33. The purity is checked with thin layered chromatography on PEI cellulose matrix developed with 0.4M ammonium bicarbonate solution.

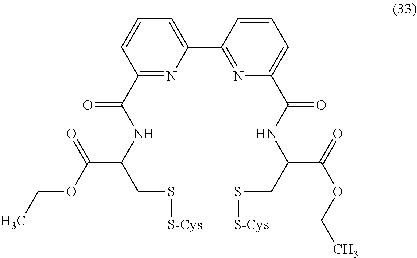

### Compound 34

2,2′-bipyridine-6,6′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 34.

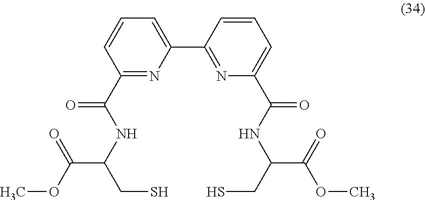

### Compound 35

Compound 34 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 35. The purity is checked with thin layered chromatography on PEI cellulose matrix developed with 0.4M ammonium bicarbonate solution.

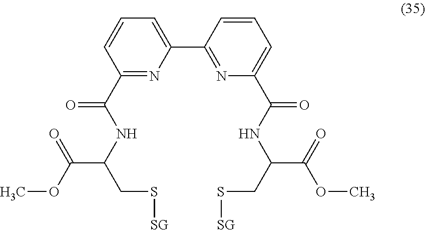

### Compound 36

Compound 34 (0.478 gm, 1 mmol) is dissolved in 10 ml of Dimethyl formamide and 5 ml of water is added to the mixture. L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 36. The purity is checked with thin layered chromatography on PEI cellulose matrix developed with 0.4M ammonium bicarbonate solution.

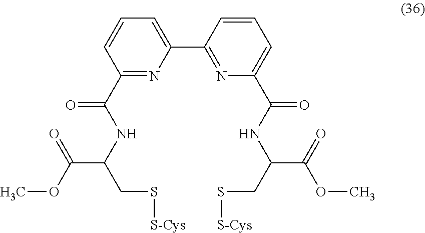

Compounds 37-45

Starting Material: 2,2′-biquinoline-4,4′-dicarboxylic acid. Aldrich Catalog number: 457191. M.W: 244.20

### Compound 37

2,2′-biquinoline-4,4′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and stirred for 12 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 37.

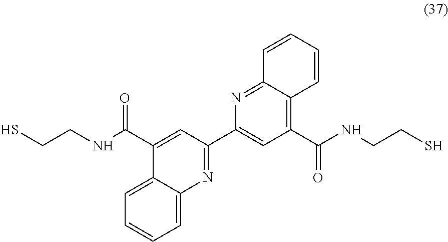 [SMILES: O=C(NCCS)c1cc(-c2cc(C(=O)NCCS)c3ccccc3n2)nc2ccccc12]

### Compound 38

Compound 37 (0.462 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 38. The purity is checked with thin layered chromatography on PEI cellulose matrix developed with 0.4M ammonium bicarbonate solution.

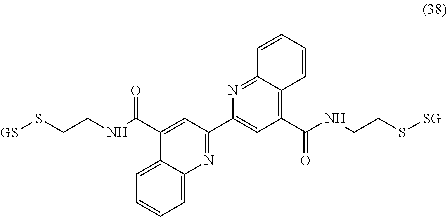

### Compound 39

Compound 37 (0.462 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 39. The purity is checked with thin layered chromatography on PEI cellulose matrix developed with 0.4M ammonium bicarbonate solution.

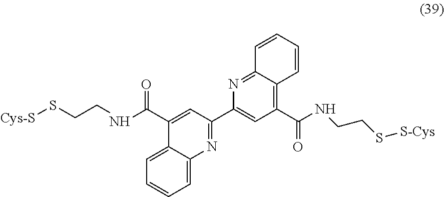

### Compound 40

2,2′-biquinoline-4,4′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and stirred for 12 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 40.

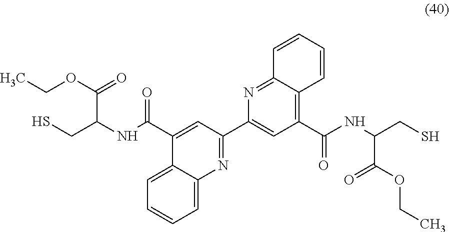 [SMILES: CCOC(=O)C(CS)NC(=O)c1cc(-c2cc(C(=O)NC(CS)C(=O)OCC)c3ccccc3n2)nc2ccccc12]

### Compound 41

Compound 40 (0.606 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 41. The purity is checked with thin layered chromatography on PEI cellulose matrix developed with 0.4M ammonium bicarbonate solution.

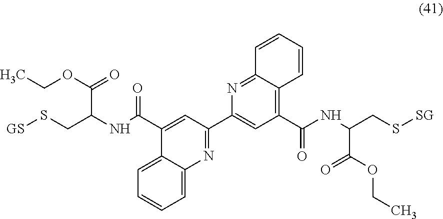

### Compound 42

Compound 40 (0.606 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 42. The purity is checked with thin layered chromatography on PEI cellulose matrix developed with 0.4M ammonium bicarbonate solution.

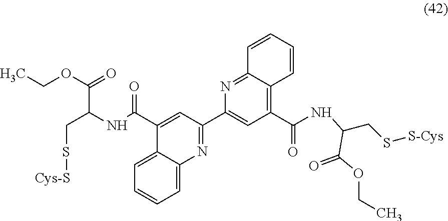

### Compound 43

2,2′-biquinoline-4,4′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and stirred for 12 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 43.

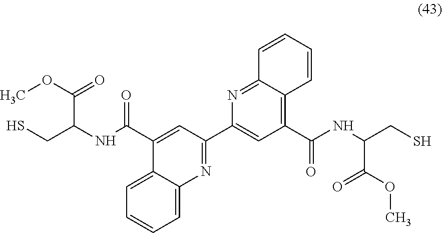 [SMILES: COC(=O)C(CS)NC(=O)c1cc(-c2cc(C(=O)NC(CS)C(=O)OC)c3ccccc3n2)nc2ccccc12]

### Compound 44

Compound 43 (0.578 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 44. The purity is checked with thin layered chromatography on PEI cellulose matrix developed with 0.4M ammonium bicarbonate solution.

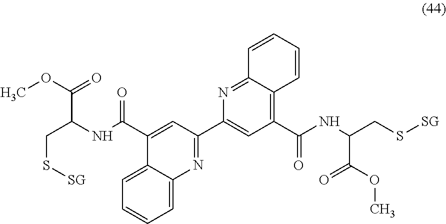

### Compound 45

Compound 43 (0.578 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 45. The purity is checked with thin layered chromatography on PEI cellulose matrix developed with 0.4M ammonium bicarbonate solution.

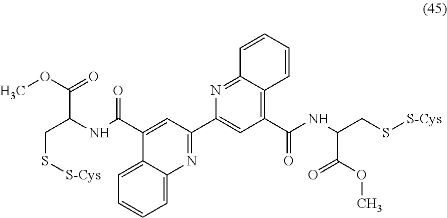

Compounds: 46-54

Starting Material: 3,4-dihydroxythiophene-2,5-dicarboxylic acid diethyl ester. Aldrich Catalog number 553174. M.W: 260.26

### Compound 46

3,4-dihydroxythiophene-2,5-dicarboxylic acid diethyl ester. (2.60 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 46.

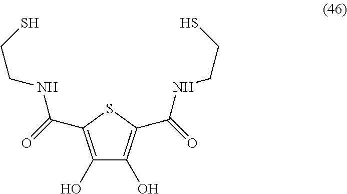 [SMILES: O=C(NCCS)c1sc(C(=O)NCCS)c(O)c1O]

### Compound 47

Compound 46 (0.322 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 47. The purity is checked with thin layered chromatography on PEI cellulose matrix developed with 0.4M ammonium bicarbonate solution.

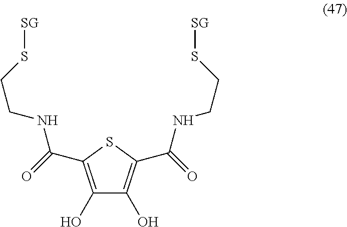

### Compound 48

Compound 46 (0.322 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 48. The purity is checked with thin layered chromatography on PEI cellulose matrix developed with 0.4M ammonium bicarbonate solution.

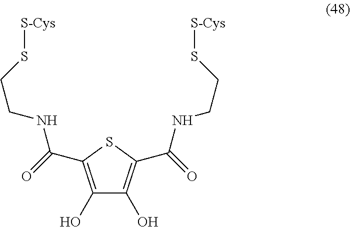

### Compound 49

3,4-dihydroxythiophene-2,5-dicarboxylic acid diethyl ester. (2.60 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 49

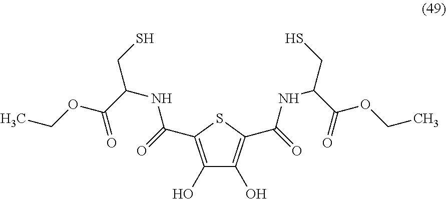 [SMILES: CCOC(=O)C(CS)NC(=O)c1sc(C(=O)NC(CS)C(=O)OCC)c(O)c1O]

### Compound 50

Compound 49 (0.466 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 50. The purity is checked with thin layered chromatography on PEI cellulose matrix developed with 0.4M ammonium bicarbonate solution.

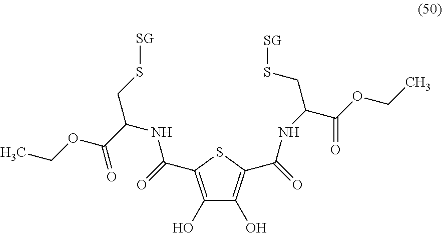

### Compound 51

Compound 49 (0.466 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 51. The purity is checked with thin layered chromatography on PEI cellulose matrix developed with 0.4M ammonium bicarbonate solution.

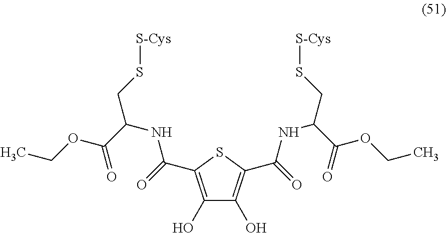

### Compound 52

3,4-dihydroxythiophene-2,5-dicarboxylic acid diethyl ester. (2.60 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 52

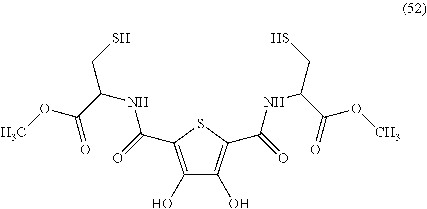

### Compound 53

Compound 52 (0.438 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 53. The purity is checked with thin layered chromatography on PEI cellulose matrix developed with 0.4M ammonium bicarbonate solution.

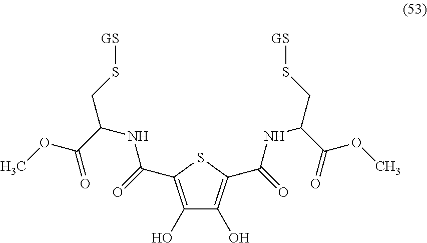

### Compound 54

Compound 52 (0.438 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 54. The purity is checked with thin layered chromatography on PEI cellulose matrix developed with 0.4M ammonium bicarbonate solution.

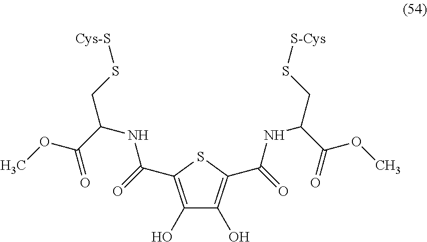

Compounds: 55-63

Starting Material: 3,4-dihydroxypyrrole-2,5-dicarboxylic acid. Aldrich Catalog number 553174. M.W: 187.10

### Compound 55

3,4-dihydroxypyrrole-2,5-dicarboxylic acid (1.87 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 55.

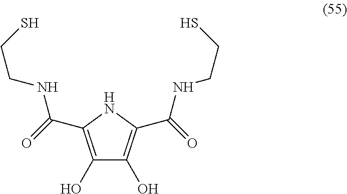 [SMILES: O=C(NCCS)c1[nH]c(C(=O)NCCS)c(O)c1O]

### Compound 56

Compound 55 (0.30 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 56. The purity is checked with thin layered chromatography on PEI cellulose matrix developed with 0.4M ammonium bicarbonate solution.

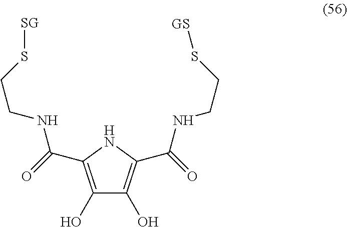

### Compound 57

Compound 55 (0.30 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 57. The purity is checked with thin layered chromatography on PEI cellulose matrix developed with 0.4M ammonium bicarbonate solution.

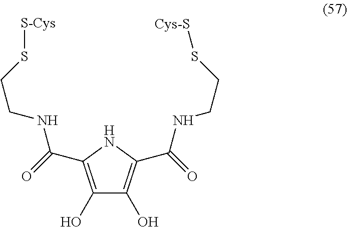

### Compound 58

3,4-dihydroxypyrrole-2,5-dicarboxylic acid (1.87 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 58.

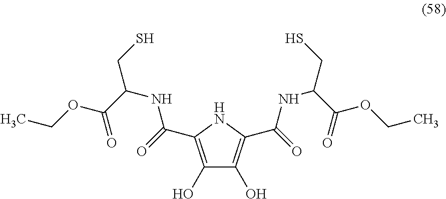 [SMILES: CCOC(=O)C(CS)NC(=O)c1[nH]c(C(=O)NC(CS)C(=O)OCC)c(O)c1O]

### Compound 59

Compound 58 (0.449 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 59. The purity is checked with thin layered chromatography on PEI cellulose matrix developed with 0.4M ammonium bicarbonate solution.

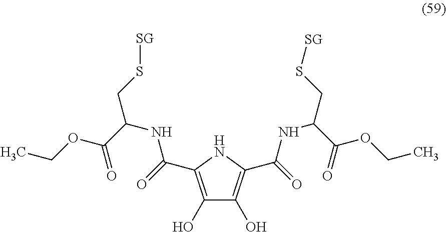

### Compound 60

Compound 58 (0.449 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 60. The purity is checked with thin layered chromatography on PEI cellulose matrix developed with 0.4M ammonium bicarbonate solution.

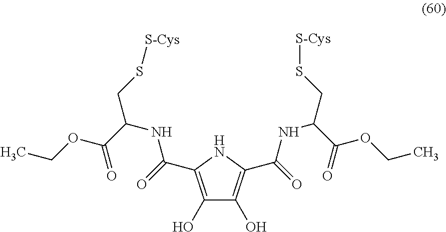

### Compound 61

3,4-dihydroxypyrrole-2,5-dicarboxylic acid (1.87 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 61.

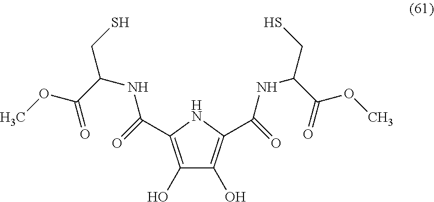

### Compound 62

Compound 61 (0.42 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 62. The purity is checked with thin layered chromatography on PEI cellulose matrix developed with 0.4M ammonium bicarbonate solution.

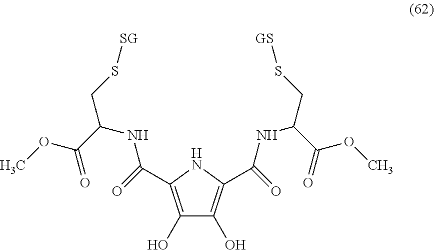

### Compound 63

Compound 61 (0.42 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 63. The purity is checked with thin layered chromatography on PEI cellulose matrix developed with 0.4M ammonium bicarbonate solution.

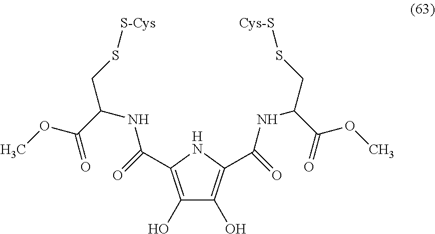

Compounds 64-72

Starting Material: 3,4-Ethylenedioxypyrrole-2,5-dicarboxylic acid. Aldrich Catalog number 637203. M.W: 223.50

### Compound 64

3,4-ethylenedioxypyrrole-2,5-dicarboxylic acid (2.23 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 64.

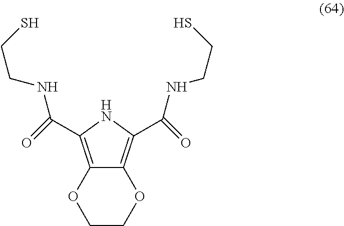 [SMILES: O=C(NCCS)c1[nH]c(C(=O)NCCS)c2c1OCCO2]

### Compound 65

Compound 64 (0.331 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 65. The purity is checked with thin layered chromatography on PEI cellulose matrix developed with 0.4M ammonium bicarbonate solution.

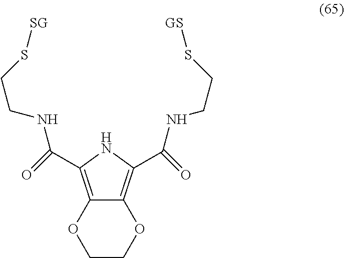

### Compound 66

Compound 64 (0.331 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 66. The purity is checked with thin layered chromatography on PEI cellulose matrix developed with 0.4M ammonium bicarbonate solution.

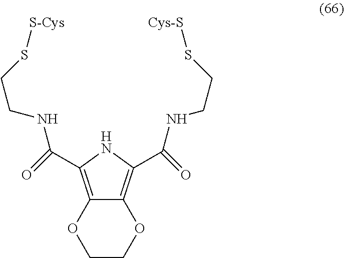

### Compound 67

3,4-Ethylenedioxypyrrole-2,5-dicarboxylic acid (2.23 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 67.

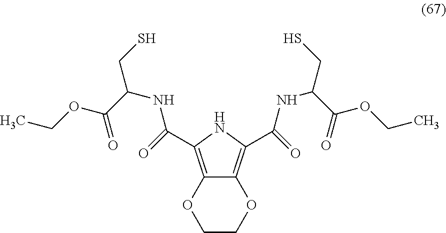 [SMILES: CCOC(=O)C(CS)NC(=O)c1[nH]c(C(=O)NC(CS)C(=O)OCC)c2c1OCCO2]

### Compound 68

Compound 67 (0.475 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 68. The purity is checked with thin layered chromatography on PEI cellulose matrix developed with 0.4M ammonium bicarbonate solution.

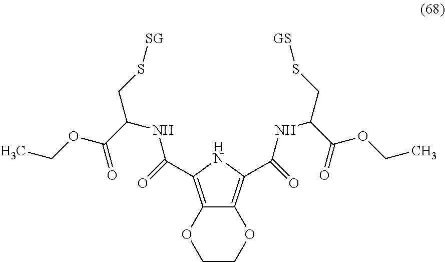

### Compound 69

Compound 67 (0.475 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 69. The purity is checked with thin layered chromatography on PEI cellulose matrix developed with 0.4M ammonium bicarbonate solution.

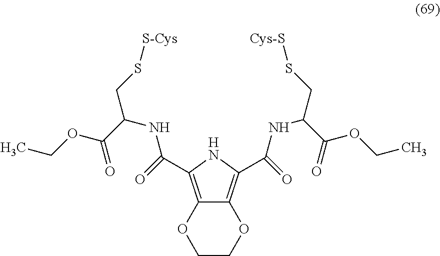

### Compound 70

3,4-Ethylenedioxypyrrole-2,5-dicarboxylic acid (2.23 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 70.

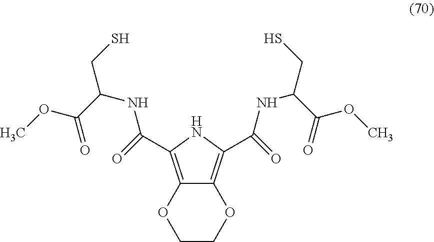

### Compound 71

Compound 70 (0.447 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 71. The purity is checked with thin layered chromatography on PEI cellulose matrix developed with 0.4M ammonium bicarbonate solution.

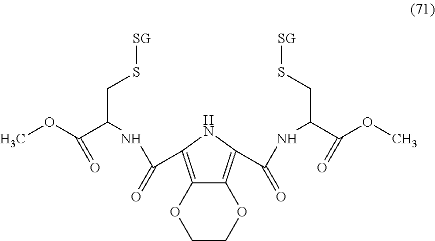

### Compound 72

Compound 70 (0.447 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 72. The purity is checked with thin layered chromatography on PEI cellulose matrix developed with 0.4M ammonium bicarbonate solution.

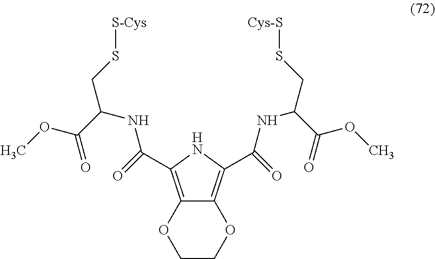

Compounds 73-81

Starting Material: 3,4-Propylenedioxythiophene-2,5-dicarboxylic acid. Aldrich Catalog number 660477. M.W: 244.22

### Compound 73

3,4-Propylenedioxythiophene-2,5-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 73.

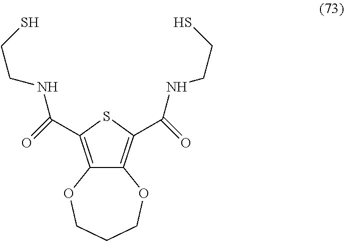 [SMILES: O=C(NCCS)c1sc(C(=O)NCCS)c2c1OCCCO2]

### Compound 74

Compound 73 (0.362 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 74. The purity is checked with thin layered chromatography on PEI cellulose matrix developed with 0.4M ammonium bicarbonate solution.

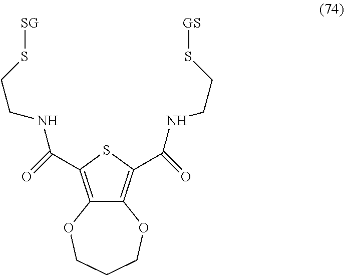

### Compound 75

Compound 73 (0.362 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 75. The purity is checked with thin layered chromatography on PEI cellulose matrix developed with 0.4M ammonium bicarbonate solution.

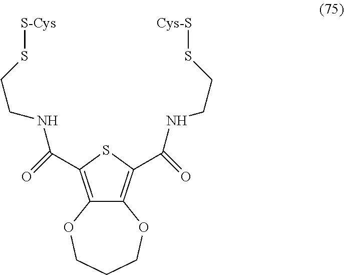

### Compound 76

3,4-Propylenedioxythiophene-2,5-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 76.

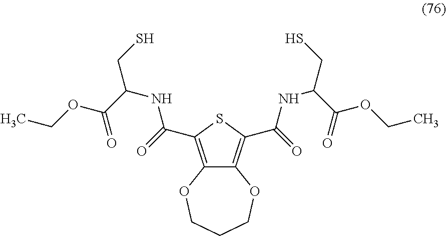 [SMILES: CCOC(=O)C(CS)NC(=O)c1sc(C(=O)NC(CS)C(=O)OCC)c2c1OCCCO2]

### Compound 77

Compound 76 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 77. The purity is checked with thin layered chromatography on PEI cellulose matrix developed with 0.4M ammonium bicarbonate solution.

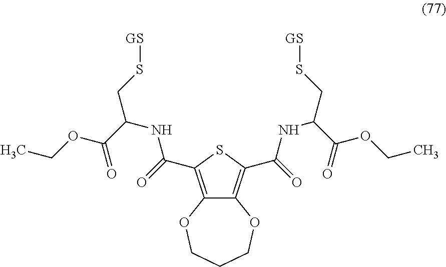

### Compound 78

Compound 76 (0.4478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 78. The purity is checked with thin layered chromatography on PEI cellulose matrix developed with 0.4M ammonium bicarbonate solution.

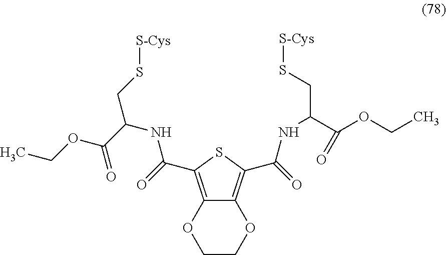

### Compound 79

3,4-Propylenedioxythiophene-2,5-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 79.

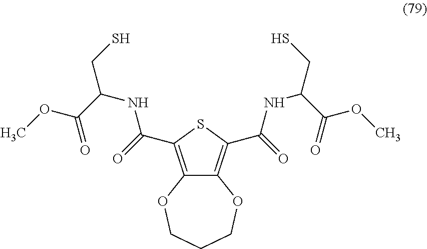

### Compound 80

Compound 79 (0.506 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 80. The purity is checked with thin layered chromatography on PEI cellulose matrix developed with 0.4M ammonium bicarbonate solution.

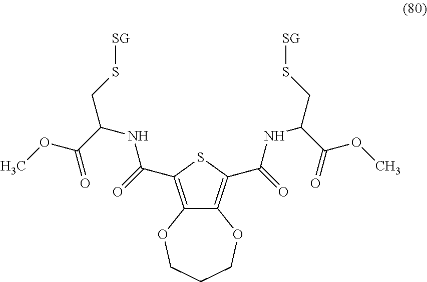

### Compound 81

Compound 79 (0.506 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 81. The purity is checked with thin layered chromatography on PEI cellulose matrix developed with 0.4M ammonium bicarbonate solution.

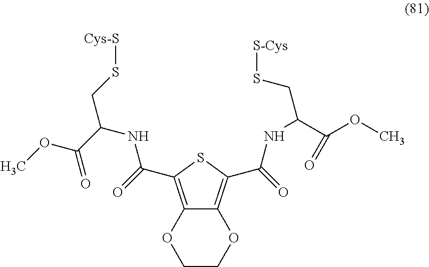

Compounds 82-90

Starting Material: 3,4-ethylenedioxythiophene-2,5-dicarboxylic acid. CAS number 108347-23.5 M.W: 230.20

### Compound 82

3,4-ethylenedioxythiophene-2,5-dicarboxylic acid (2.30 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 82.

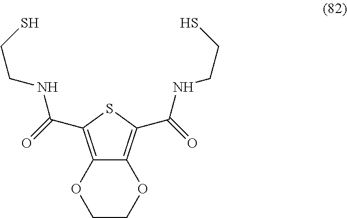 [SMILES: O=C(NCCS)c1sc(C(=O)NCCS)c2c1OCCO2]

### Compound 83

Compound 82 (0.348 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 83. The purity is checked with thin layered chromatography on PEI cellulose matrix developed with 0.4M ammonium bicarbonate solution.

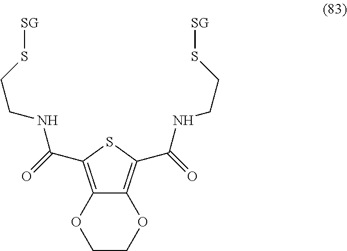

### Compound 84

Compound 82 (0.348 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 84. The purity is checked with thin layered chromatography on PEI cellulose matrix developed with 0.4M ammonium bicarbonate solution.

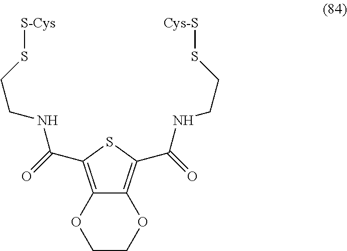

### Compound 85

3,4-ethylenedioxythiophene-2,5-dicarboxylic acid (2.30 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 85.

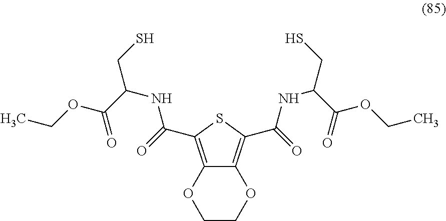 [SMILES: CCOC(=O)C(CS)NC(=O)c1sc(C(=O)NC(CS)C(=O)OCC)c2c1OCCO2]

### Compound 86

Compound 85 (0.492 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 86. The purity is checked with thin layered chromatography on PEI cellulose matrix developed with 0.4M ammonium bicarbonate solution.

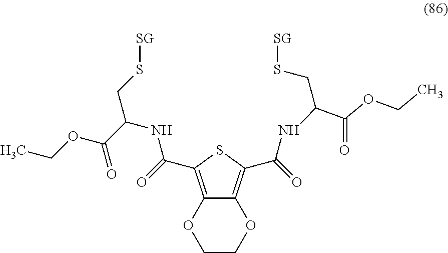

### Compound 87

Compound 85 (0.492 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 87. The purity is checked with thin layered chromatography on PEI cellulose matrix developed with 0.4M ammonium bicarbonate solution.

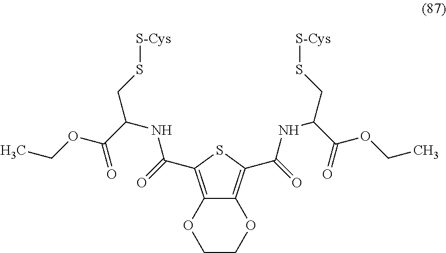

### Compound 88

3,4-ethylenedioxythiophene-2,5-dicarboxylic acid (2.30 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 88.

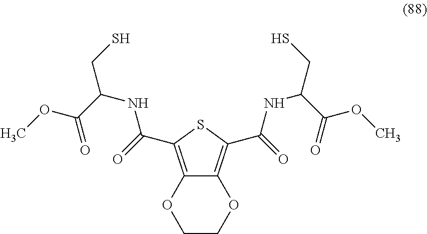

### Compound 89

Compound 88 (0.464 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 89. The purity is checked with thin layered chromatography on PEI cellulose matrix developed with 0.4M ammonium bicarbonate solution.

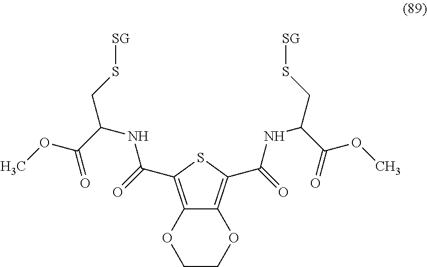

### Compound 90

Compound 88 (0.464 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 90. The purity is checked with thin layered chromatography on PEI cellulose matrix developed with 0.4M ammonium bicarbonate solution.

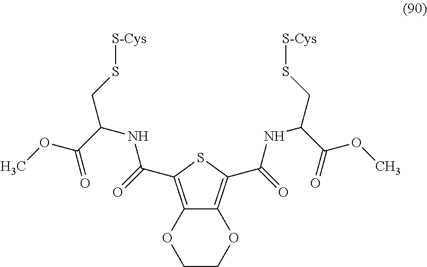

Compounds 91-99

Starting Material: 3,4-Propylenedioxypyrrole-2,5-dicarboxylic acid. Aldrich Catalog number 637432. M.W: 227.17

### Compound 91

3,4-propylenedioxypyrrole-2,5-dicarboxylic acid (2.27 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 91.

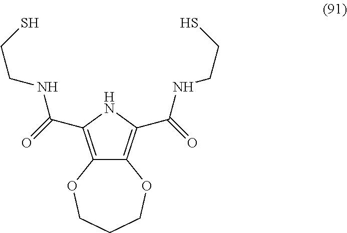 [SMILES: O=C(NCCS)c1[nH]c(C(=O)NCCS)c2c1OCCCO2]

### Compound 92

Compound 91 (0.345 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 92. The purity is checked with thin layered chromatography on PEI cellulose matrix developed with 0.4M ammonium bicarbonate solution.

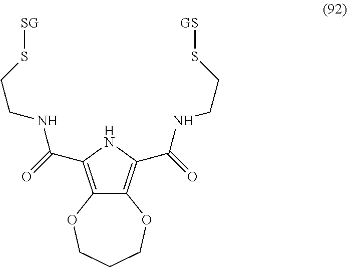

### Compound 93

Compound 91 (0.345 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 93. The purity is checked with thin layered chromatography on PEI cellulose matrix developed with 0.4M ammonium bicarbonate solution.

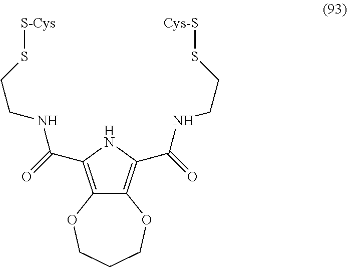

### Compound 94

3,4-Propylenedioxypyrrole-2,5-dicarboxylic acid (2.27 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 94.

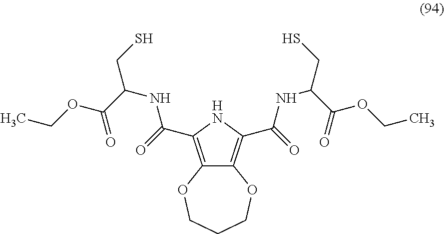 [SMILES: CCOC(=O)C(CS)NC(=O)c1[nH]c(C(=O)NC(CS)C(=O)OCC)c2c1OCCCO2]

### Compound 95

Compound 94 (0.489 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 95. The purity is checked with thin layered chromatography on PEI cellulose matrix developed with 0.4M ammonium bicarbonate solution.

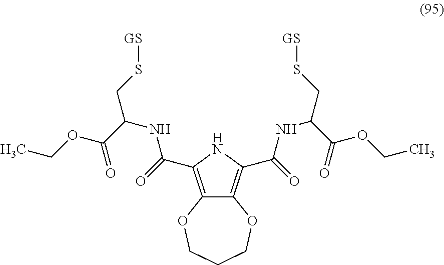

### Compound 96

Compound 94 (0.489 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 96. The purity is checked with thin layered chromatography on PEI cellulose matrix developed with 0.4M ammonium bicarbonate solution.

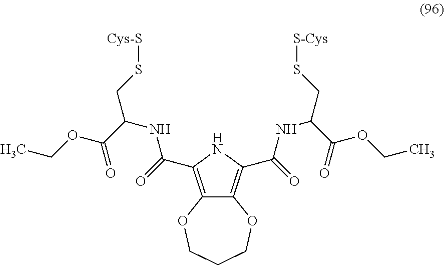

### Compound 97

3,4-Propylenedioxypyrrole-2,5-dicarboxylic acid (2.27 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 97.

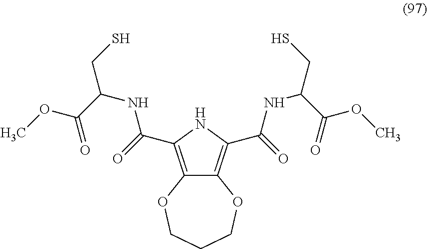

### Compound 98

Compound 97 (0.461 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 98. The purity is checked with thin layered chromatography on PEI cellulose matrix developed with 0.4M ammonium bicarbonate solution.

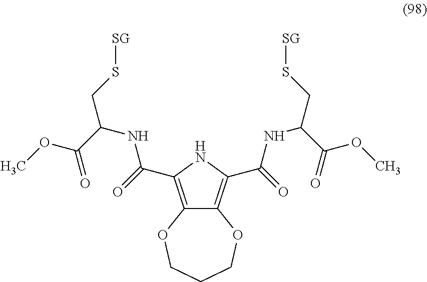

### Compound 99

Compound 97 (0.461 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 99. The purity is checked with thin layered chromatography on PEI cellulose matrix developed with 0.4M ammonium bicarbonate solution.

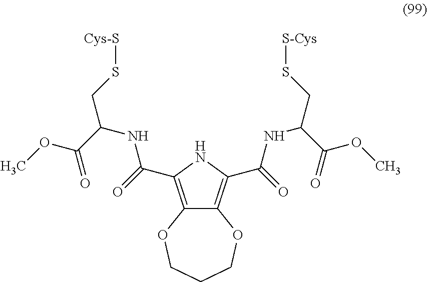

### Compound 100-108

Starting Material: Biphenyl-2,2′-dicarboxylic acid. Aldrich Catalog number: 126691. M.W: 242.23

### Compound 100

Biphenyl-2,2′-dicarboxylic acid (2.42 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 100.

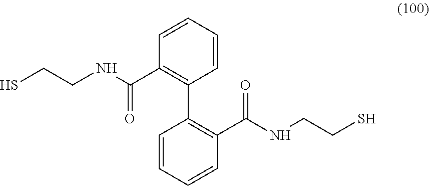 [SMILES: O=C(NCCS)c1ccccc1-c1ccccc1C(=O)NCCS]

### Compound 101

Compound 100 (0.360 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 101. The purity is checked with thin layered chromatography on PEI cellulose matrix developed with 0.4M ammonium bicarbonate solution.

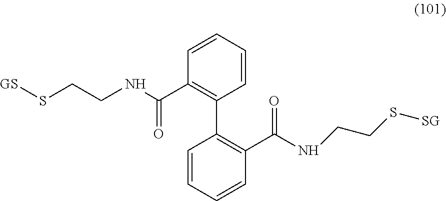

### Compound 102

Compound 100 (0.362 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 102. The purity is checked with thin layered chromatography on PEI cellulose matrix developed with 0.4M ammonium bicarbonate solution.

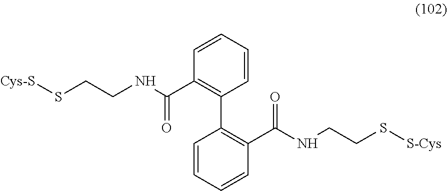

### Compound 103

2 Biphenyl-2,2′-dicarboxylic acid (2.42 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 103.

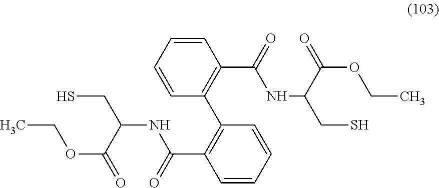 [SMILES: CCOC(=O)C(CS)NC(=O)c1ccccc1-c1ccccc1C(=O)NC(CS)C(=O)OCC]

### Compound 104

Compound 103 (0.506 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 104. The purity is checked with thin layered chromatography on PEI cellulose matrix developed with 0.4M ammonium bicarbonate solution.

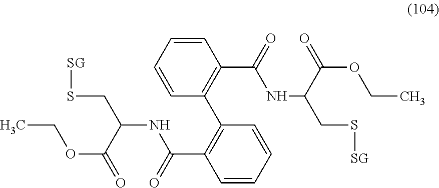

### Compound 105

Compound 103 (0.506 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 105. The purity is checked with thin layered chromatography on PEI cellulose matrix developed with 0.4M ammonium bicarbonate solution.

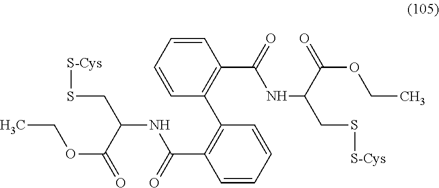

### Compound 106

Biphenyl-2,2′-dicarboxylic acid (2.42 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 106.

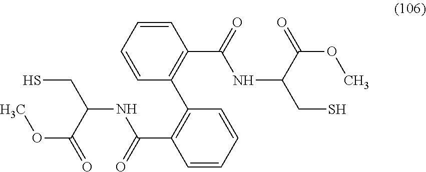

### Compound 107

Compound 106 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 107. The purity is checked with thin layered chromatography on PEI cellulose matrix developed with 0.4M ammonium bicarbonate solution.

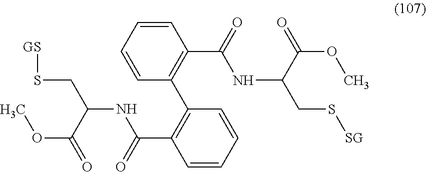

### Compound 108

Compound 106 (0.478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 108. The purity is checked with thin layered chromatography on PEI cellulose matrix developed with 0.4M ammonium bicarbonate solution.

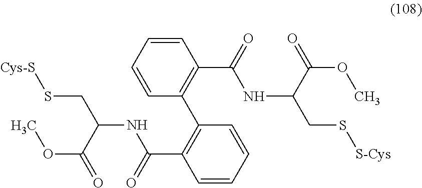

Compounds 109-117

Starting Material: Chelidonic acid or 4-oxo-4H-pyran-2,6-dicarboxylic acid. Fluka Catalog number 22500. M.W: 184.10

### Compound 109

Chelidonic acid (1.84 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 109.

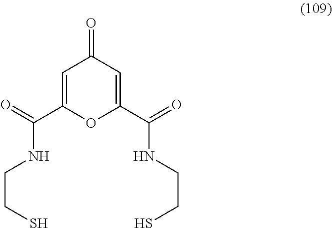 [SMILES: O=C(NCCS)c1cc(=O)cc(C(=O)NCCS)o1]

### Compound 110

Compound 109 (0.300 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 110. The purity is checked with thin layered chromatography on PEI cellulose matrix developed with 0.4M ammonium bicarbonate solution.

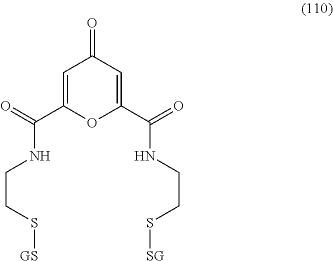

### Compound 111

Compound 109 (0.300 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 111. The purity is checked with thin layered chromatography on PEI cellulose matrix developed with 0.4M ammonium bicarbonate solution.

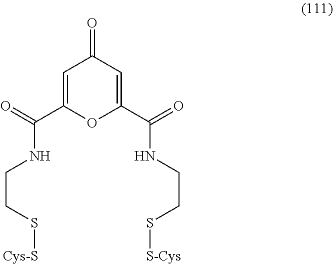

### Compound 112

Chelidonic acid (1.84 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 112.

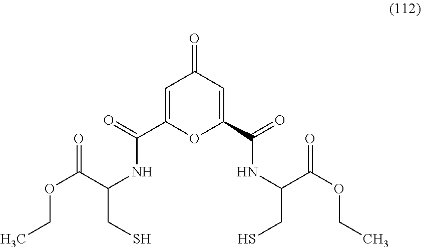

### Compound 113

Compound 112 (0.444 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 113. The purity is checked with thin layered chromatography on PEI cellulose matrix developed with 0.4M ammonium bicarbonate solution.

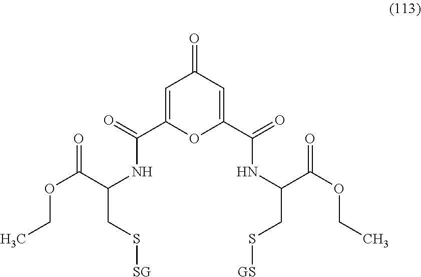

### Compound 114

Compound 112 (0.444 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 114. The purity is checked with thin layered chromatography on PEI cellulose matrix developed with 0.4M ammonium bicarbonate solution.

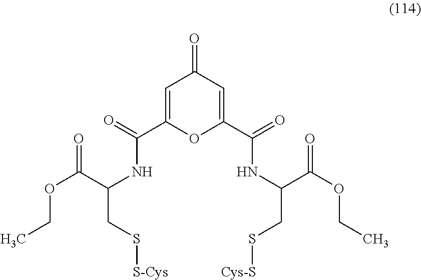

### Compound 115

Chelidonic acid (1.84 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 115.

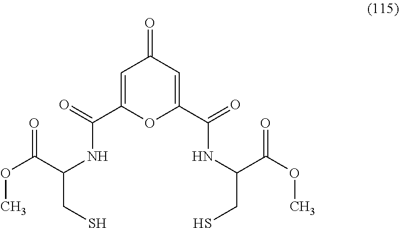 [SMILES: COC(=O)C(CS)NC(=O)c1cc(=O)cc(C(=O)NC(CS)C(=O)OC)o1]

### Compound 116

Compound 115 (0.416 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 116. The purity is checked with thin layered chromatography on PEI cellulose matrix developed with 0.4M ammonium bicarbonate solution.

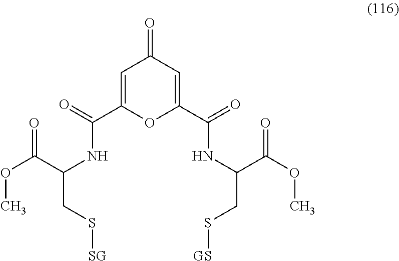

### Compound 117

Compound 115. (0.416 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 117. The purity is

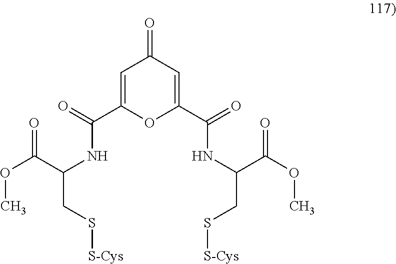

Compounds 118-126

Starting Material: Terephthaloyl dichloride. Aldrich Cat #120871 Mol wt: 203.02

### Compound 118

Cysteamine hydrochloride (2.825 gm, 25 mmol) is dissolved in a 100 ml round bottomed flask and chloroform (50 ml) is added to it and stirred. Triethylamine (3.1 gm, 30 mmol) is added to it drop by drop under constant stirring. Once all the cysteamine hydrochloride has dissolved terephthaloyl dichloride (2.03 g, 10 mmol) dissolved in 20 ml chloroform is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 118.

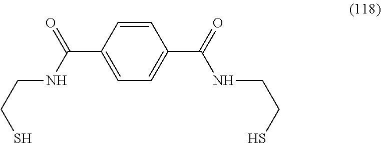 [SMILES: O=C(NCCS)c1ccc(C(=O)NCCS)cc1]

### Compound 119

Compound 118 (0.284 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 129. The purity is checked with thin layered chromatography on PEI cellulose matrix developed with 0.4M ammonium bicarbonate solution.

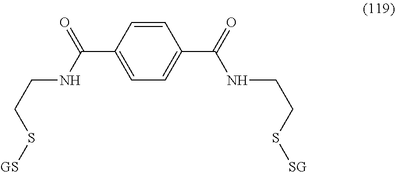

### Compound 120

Compound 118 (0.284 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 120. The purity is checked with thin layered chromatography on PEI cellulose matrix developed with 0.4M ammonium bicarbonate solution.

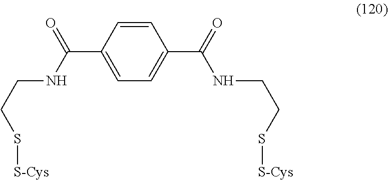

### Compound 121

L-cysteine hydrochloride ethyl ester (2.825 gm, 25 mmol) is dissolved in a 100 ml round bottomed flask and chloroform (50 ml) is added to it and stirred. Triethylamine (3.1 gm, 30 mmol) is added to it drop by drop under constant stirring. Once all the L-cysteine hydrochloride ethyl ester has dissolved terephthaloyl dichloride (2.03 g, 10 mmol) dissolved in 20 ml chloroform is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 121.

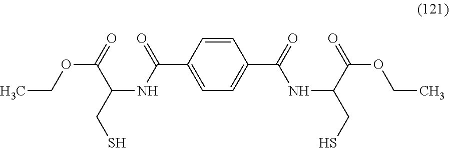 [SMILES: CCOC(=O)C(CS)NC(=O)c1ccc(C(=O)NC(CS)C(=O)OCC)cc1]

### Compound 122

Compound 121 (0.428 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 122. The purity is checked with thin layered chromatography on PEI cellulose matrix developed with 0.4M ammonium bicarbonate solution.

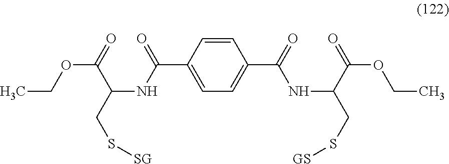

### Compound 123

Compound 121 (0.469 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 123. The purity is

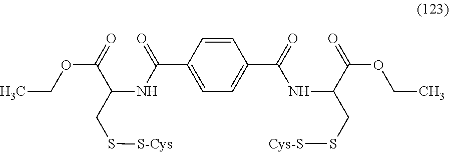

### Compound 124

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved terephthaloyl dichloride (2.03 g, 10 mmol) dissolved in 20 ml chloroform is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 124.

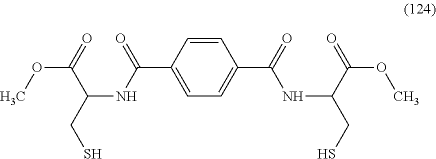

### Compound 125

Compound 124 (0.400 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 125. The purity is checked with thin layered chromatography on PEI cellulose matrix developed with 0.4M ammonium bicarbonate solution.

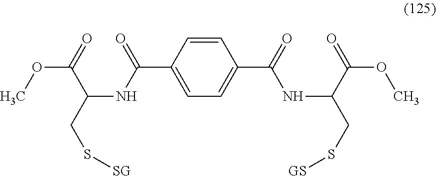

### Compound 126

Compound 124 (0.400 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 126. The purity is checked with thin layered chromatography on PEI cellulose matrix developed with 0.4M ammonium bicarbonate solution.

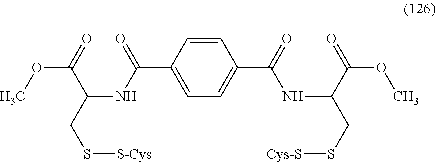

Compounds 127-135

Starting Material: Homophthalic acid. Aldrich Catalog number: P-63603. M.W: 180.16

### Compound 127

Homophthalic acid (1.80 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 127.

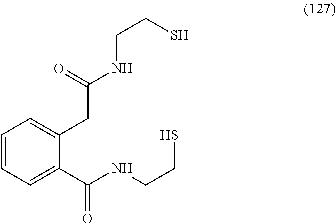 [SMILES: O=C(Cc1ccccc1C(=O)NCCS)NCCS]

### Compound 128

Compound 127 (0.298 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 128. The purity is checked with thin layered chromatography on PEI cellulose matrix developed with 0.4M ammonium bicarbonate solution.

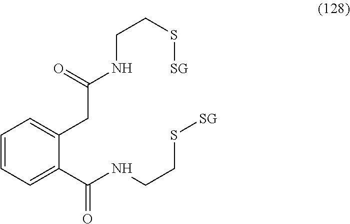

### Compound 129

Compound 127 (0.298 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 129. The purity is

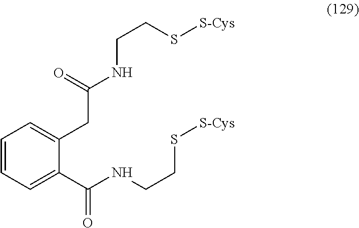

### Compound 130

Homophthalic acid (1.80 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 130.

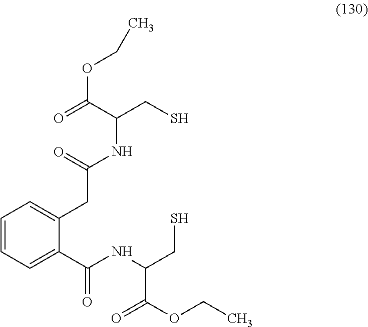 [SMILES: CCOC(=O)C(CS)NC(=O)Cc1ccccc1C(=O)NC(CS)C(=O)OCC]

### Compound 131

Compound 130 (0.442 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 131. The purity is checked with thin layered chromatography on PEI cellulose matrix developed with 0.4M ammonium bicarbonate solution.

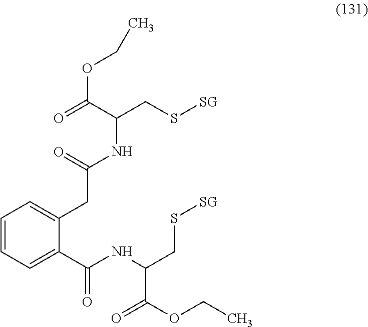

### Compound 132

Compound 130 (0.442 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 132. The purity is

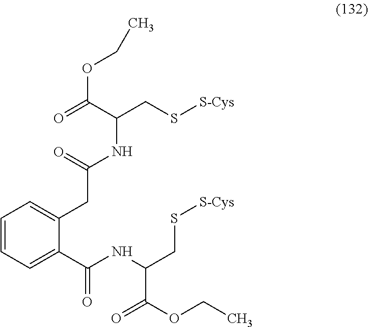

### Compound 133

Homophthalic acid (1.80 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 133.

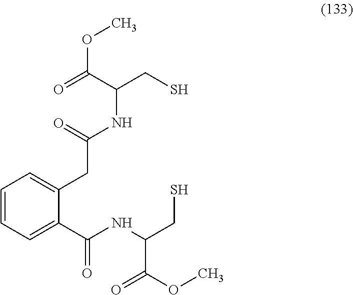 [SMILES: COC(=O)C(CS)NC(=O)Cc1ccccc1C(=O)NC(CS)C(=O)OC]

### Compound 134

Compound 133 (0.414 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 134. The purity is checked with thin layered chromatography on PEI cellulose matrix developed with 0.4M ammonium bicarbonate solution.

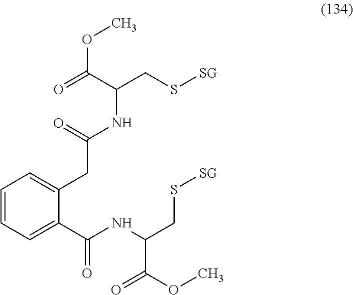

### Compound 135

Compound 133 (0.414 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 135. The purity is checked with thin layered chromatography on PEI cellulose matrix developed with 0.4M ammonium bicarbonate solution.

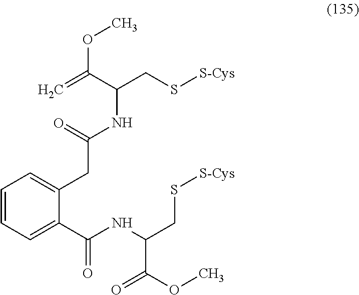

Compounds: 136-144

Starting Material: 1-Benzyl-1H-pyrazole-3,5-dicarboxylic acid. Aldrich Catalog number: S 457000. M.W: 342.35

### Compound 136

1-Benzyl-1H-pyrazole-3,5-dicarboxylic acid (3.42 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 136.

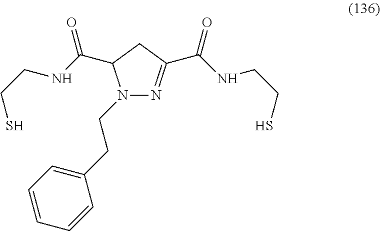 [SMILES: O=C(NCCS)C1=NN(CCc2ccccc2)C(C(=O)NCCS)C1]

### Compound 137

Compound 136 (0.38 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 137. The purity is checked with thin layered chromatography on PEI cellulose matrix developed with 0.4M ammonium bicarbonate solution.

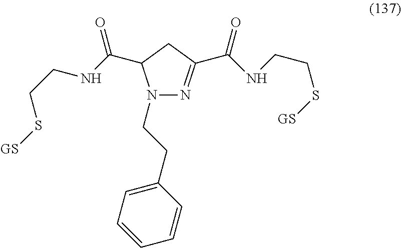

### Compound 138

Compound 136 (0.38 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 138. The purity is

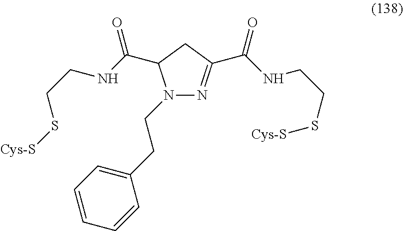

### Compound 139

1-Benzyl-1H-pyrazole-3,5-dicarboxylic acid (3.42 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 139.

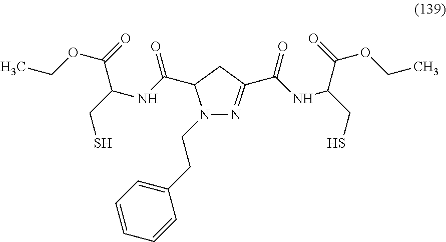 [SMILES: CCOC(=O)C(CS)NC(=O)C1=NN(CCc2ccccc2)C(C(=O)NC(CS)C(=O)OCC)C1]

### Compound 140

Compound 139 (0.524 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 140. The purity is checked with thin layered chromatography on PEI cellulose matrix developed with 0.4M ammonium bicarbonate solution.

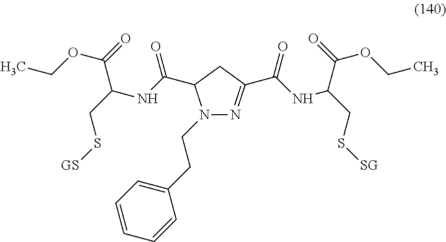

### Compound 141

Compound 139 (0.524 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 141. The purity is checked with thin layered chromatography on PEI cellulose matrix developed with 0.4M ammonium bicarbonate solution.

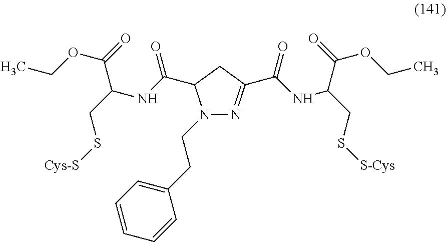

### Compound 142

1-Benzyl-1H-pyrazole-3,5-dicarboxylic acid (3.42 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 142.

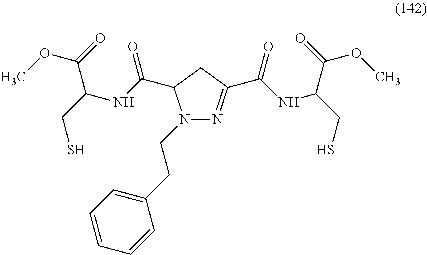

### Compound 143

Compound 142 (0.496 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 143. The purity is checked with thin layered chromatography on PEI cellulose matrix developed with 0.4M ammonium bicarbonate solution.

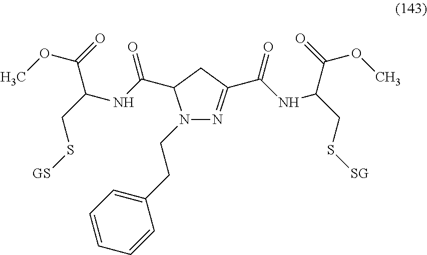

### Compound 144

Compound 142 (0.42 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 144. The purity is checked with thin layered chromatography on PEI cellulose matrix developed with 0.4M ammonium bicarbonate solution.

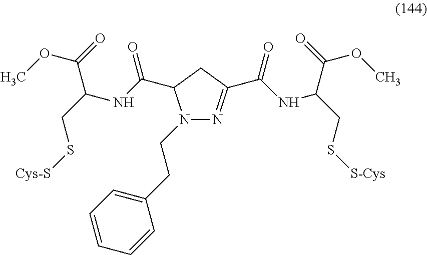

Compounds 145-153

Starting Material: 4-phenyl-pyridine-2,5-dicarboxylic acid. Aldrich Catalog number: 3-95889. M.W: 243.22

### Compound 145

4-phenyl-pyridine-2,5-dicarboxylic acid (2.43 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 145.

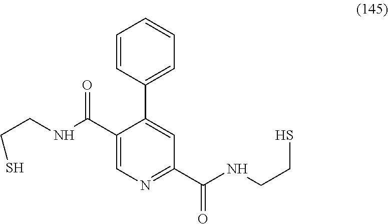 [SMILES: O=C(NCCS)c1cc(-c2ccccc2)c(C(=O)NCCS)cn1]

### Compound 146

Compound 145 (0.361 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 146. The purity is checked with thin layered chromatography on PEI cellulose matrix developed with 0.4M ammonium bicarbonate solution.

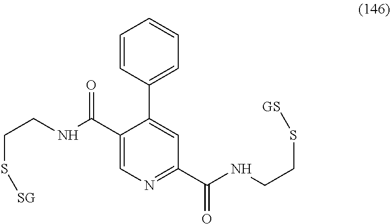

### Compound 147

Compound 145 (0.361 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 147. The purity is checked with thin layered chromatography on PEI cellulose matrix developed with 0.4M ammonium bicarbonate solution.

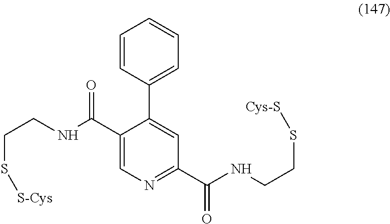

### Compound 148

4-phenyl-pyridine-2,5-dicarboxylic acid (2.43 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 148.

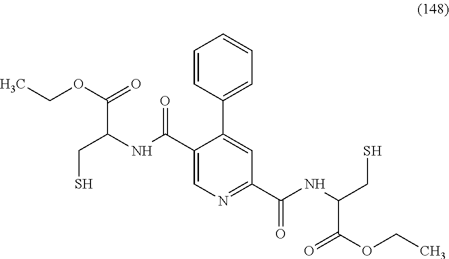 [SMILES: CCOC(=O)C(CS)NC(=O)c1cc(-c2ccccc2)c(C(=O)NC(CS)C(=O)OCC)cn1]

### Compound 149

Compound 148 (0.505 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 149. The purity is checked with thin layered chromatography on PEI cellulose matrix developed with 0.4M ammonium bicarbonate solution.

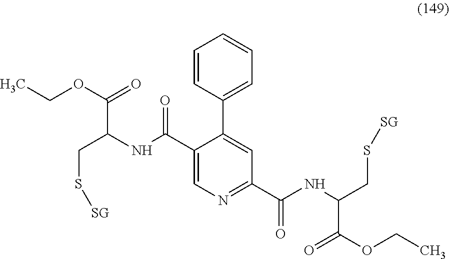

### Compound 150

Compound 148 (0.505 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 150. The purity is

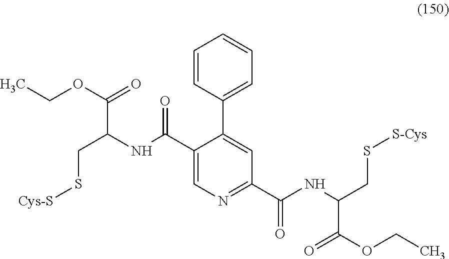

### Compound 151

4-phenyl-pyridine-2,5-dicarboxylic acid (2.43 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 151.

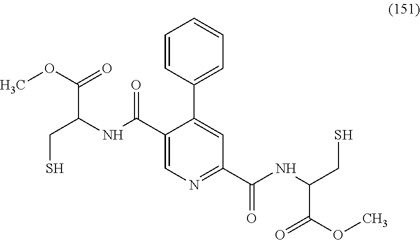

### Compound 152

Compound 151 (0.477 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 152. The purity is checked with thin layered chromatography on PEI cellulose matrix developed with 0.4M ammonium bicarbonate solution.

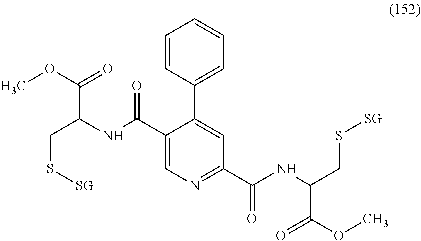

### Compound 153

Compound 151 (0.477 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 153. The purity is checked with thin layered chromatography on PEI cellulose matrix developed with 0.4M ammonium bicarbonate solution.

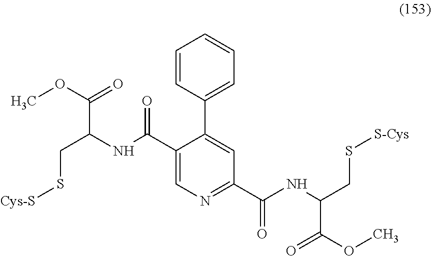

Compounds: 154-162

Starting Material: Thiophene-2,5-dicarboxylic acid dimethyl ester. Aldrich Catalog number R 416584. M.W: 200.215

### Compound 154

Thiophene-2,5-dicarboxylic acid dimethyl ester (2.00 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 154.

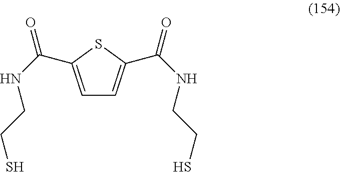 [SMILES: O=C(NCCS)c1ccc(C(=O)NCCS)s1]

### Compound 155

Compound 154 (0.29 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 155. The purity is checked with thin layered chromatography on PEI cellulose matrix developed with 0.4M ammonium bicarbonate solution.

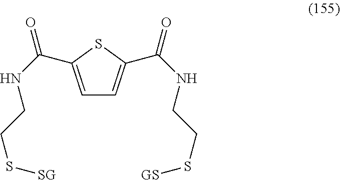

### Compound 156

Compound 154 (0.29 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 156. The purity is checked with thin layered chromatography on PEI cellulose matrix developed with 0.4M ammonium bicarbonate solution.

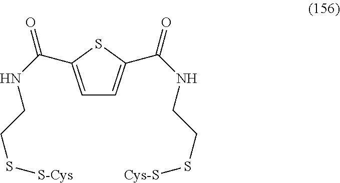

### Compound 157

Thiophene-2,5-dicarboxylic acid dimethyl ester (2.00 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 157.

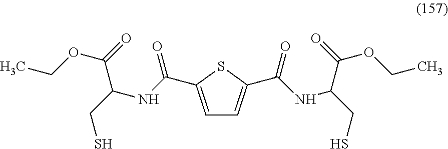 [SMILES: CCOC(=O)C(CS)NC(=O)c1ccc(C(=O)NC(CS)C(=O)OCC)s1]

### Compound 158

Compound 157 (0.434 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 158. The purity is checked with thin layered chromatography on PEI cellulose matrix developed with 0.4M ammonium bicarbonate solution.

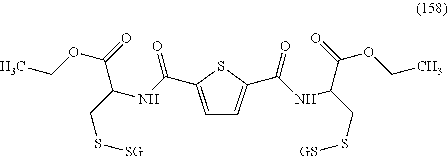

### Compound 159

Compound 157 (0.434 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 159. The purity is checked with thin layered chromatography on PEI cellulose matrix developed with 0.4M ammonium bicarbonate solution.

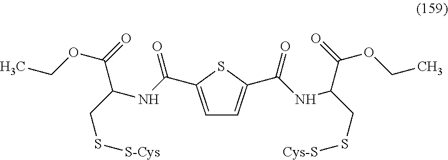

### Compound 160

Thiophene-2,5-dicarboxylic acid dimethyl ester (2.00 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 160.

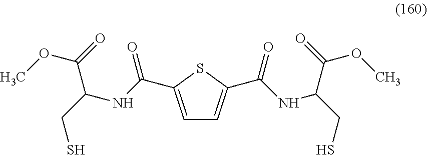

### Compound 161

Compound 160 (0.40 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 161. The purity is checked with thin layered chromatography on PEI cellulose matrix developed with 0.4M ammonium bicarbonate solution.

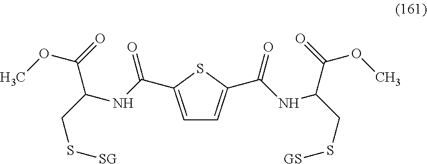

### Compound 162

Compound 160 (0.40 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 162. The purity is checked with thin layered chromatography on PEI cellulose matrix developed with 0.4M ammonium bicarbonate solution.

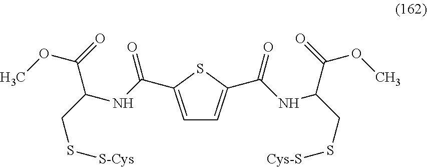

Compounds: 163-171

Starting Material: 3-Methyl thiophene-2,4-dicarboxylic acid dimethyl ester. Aldrich Catalog number R 432628. M.W: 214.24

### Compound 163

3-Methyl thiophene-2,4-dicarboxylic acid dimethyl ester (2.14 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 163.

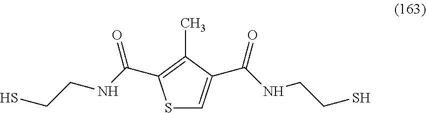 [SMILES: Cc1c(C(=O)NCCS)csc1C(=O)NCCS]

### Compound 164

Compound 163 (0.30 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 164. The purity is checked with thin layered chromatography on PEI cellulose matrix developed with 0.4M ammonium bicarbonate solution.

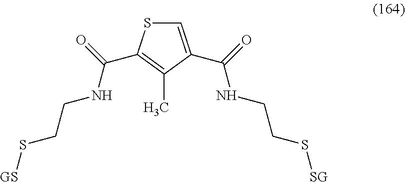

### Compound 165

Compound 163 (0.30 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 165. The purity is

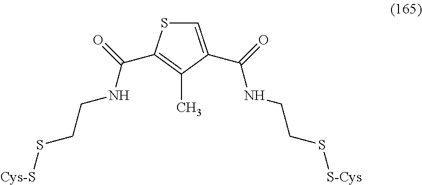

### Compound 166

3-Methyl thiophene-2,4-dicarboxylic acid dimethyl ester (2.14 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 166.

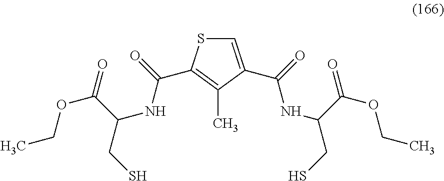 [SMILES: CCOC(=O)C(CS)NC(=O)c1csc(C(=O)NC(CS)C(=O)OCC)c1C]

### Compound 167

Compound 166 (0.448 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 167. The purity is checked with thin layered chromatography on PEI cellulose matrix developed with 0.4M ammonium bicarbonate solution.

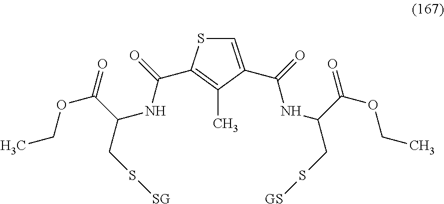

### Compound 168

Compound 166 (0.448 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 168. The purity is checked with thin layered chromatography on PEI cellulose matrix developed with 0.4M ammonium bicarbonate solution.

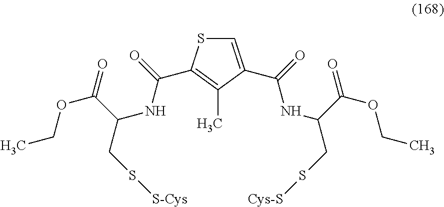

### Compound 169

3-Methyl thiophene-2,4-dicarboxylic acid dimethyl ester (2.14 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 169.

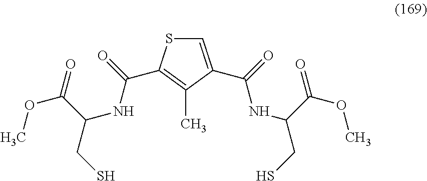

### Compound 170

Compound 169 (0.42 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 170. The purity is checked with thin layered chromatography on PEI cellulose matrix developed with 0.4M ammonium bicarbonate solution.

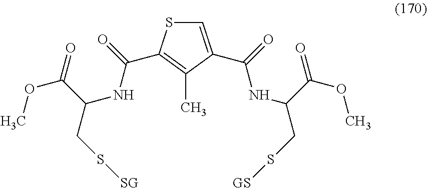

### Compound 171

Compound 169 (0.42 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 171. The purity is checked with thin layered chromatography on PEI cellulose matrix developed with 0.4M ammonium bicarbonate solution.

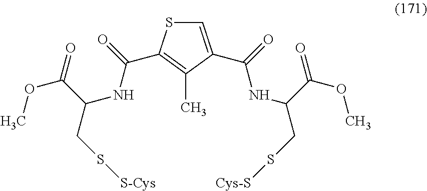

### Compound 172-180

Starting Material: Dimethyl-2,6-napthalene dicarboxylic acid. Fluka Catalog number: 70230. M.W: 244.24

### Compound 172

Dimethyl-2,6-napthalene dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 172.

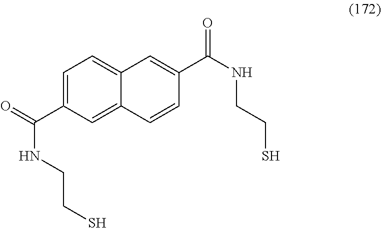 [SMILES: O=C(NCCS)c1ccc2cc(C(=O)NCCS)ccc2c1]

### Compound 173

Compound 172 (0.334 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 173. The purity is checked with thin layered chromatography on PEI cellulose matrix developed with 0.4M ammonium bicarbonate solution.

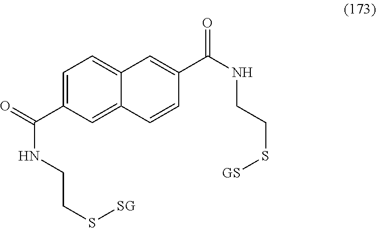

### Compound 174

Compound 172 (0.334 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 174. The purity is checked with thin layered chromatography on PEI cellulose matrix developed with 0.4M ammonium bicarbonate solution.

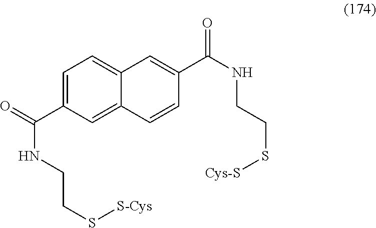

### Compound 175

Dimethyl-2,6-napthalene dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 175.

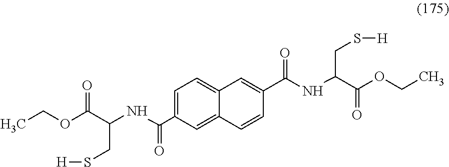 [SMILES: CCOC(=O)C(CS)NC(=O)c1ccc2cc(C(=O)NC(CS)C(=O)OCC)ccc2c1]

### Compound 176

Compound 175 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 176. The purity is checked with thin layered chromatography on PEI cellulose matrix developed with 0.4M ammonium bicarbonate solution.

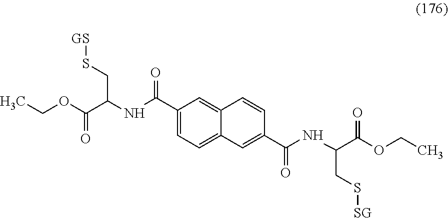

### Compound 177

Compound 175 (0.478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 177. The purity is

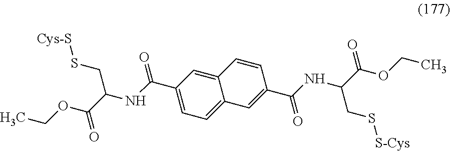

### Compound 178

Dimethyl-2,6-napthalene dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 178.

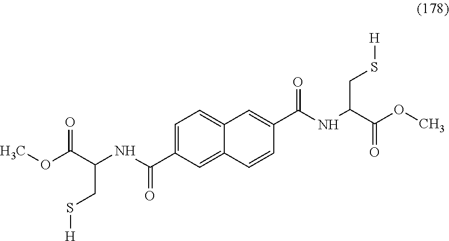

### Compound 179

Compound 178 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 179. The purity is checked with thin layered chromatography on PEI cellulose matrix developed with 0.4M ammonium bicarbonate solution.

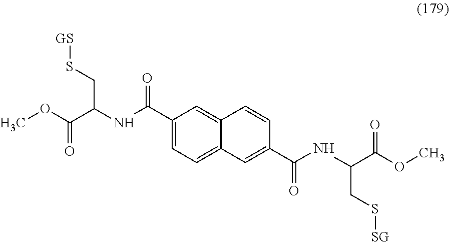

### Compound 180

Compound 178 (0.478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 180. The purity is checked with thin layered chromatography on PEI cellulose matrix developed with 0.4M ammonium bicarbonate solution.

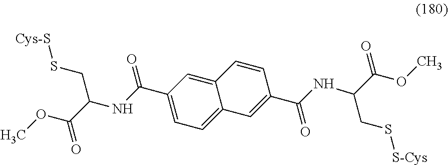

### Compound 181-189

Starting Material: 4,4′-sulfonyldibenzoic acid. Aldrich Catalog number: 163295. M.W: 306.29

### Compound 181

4,4′-sulfonyldibenzoic acid (3.06 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 181.

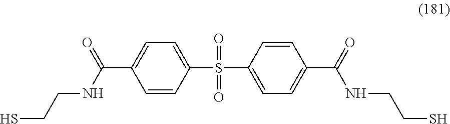 [SMILES: O=C(NCCS)c1ccc(S(=O)(=O)c2ccc(C(=O)NCCS)cc2)cc1]

### Compound 182

Compound 181 (0.424 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 182. The purity is checked with thin layered chromatography on PEI cellulose matrix developed with 0.4M ammonium bicarbonate solution.

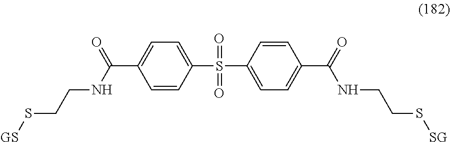

### Compound 183

Compound 181 (0.424 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 183. The purity is checked with thin layered chromatography on PEI cellulose matrix developed with 0.4M ammonium bicarbonate solution.

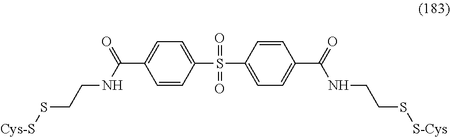

### Compound 184

4,4′-sulfonyldibenzoic acid (3.06 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 184.

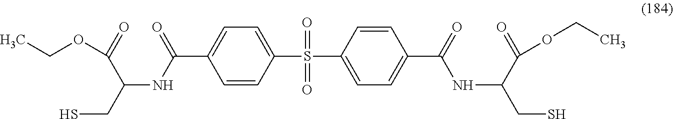 [SMILES: CCOC(=O)C(CS)NC(=O)c1ccc(S(=O)(=O)c2ccc(C(=O)NC(CS)C(=O)OCC)cc2)cc1]

### Compound 185

Compound 184 (0.568 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 185. The purity is checked with thin layered chromatography on PEI cellulose matrix developed with 0.4M ammonium bicarbonate solution.

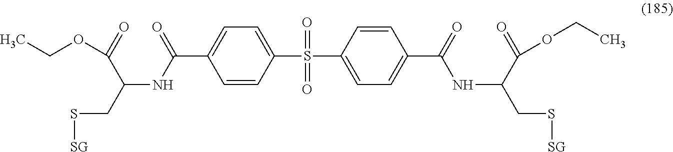

### Compound 186

Compound 184 (0.568 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 186. The purity is checked with thin layered chromatography on PEI cellulose matrix developed with 0.4M ammonium bicarbonate solution.

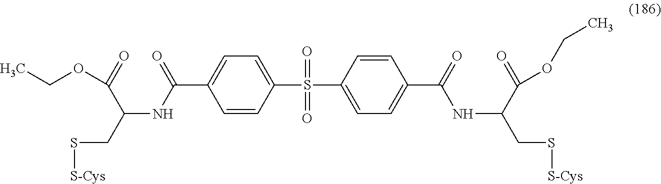

### Compound 187

4,4′-sulfonyldibenzoic acid (3.06 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 187.

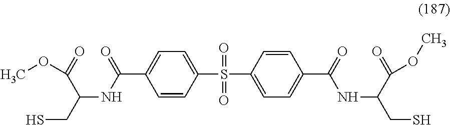

### Compound 188

Compound 187 (0.54 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 188. The purity is checked with thin layered chromatography on PEI cellulose matrix developed with 0.4M ammonium bicarbonate solution.

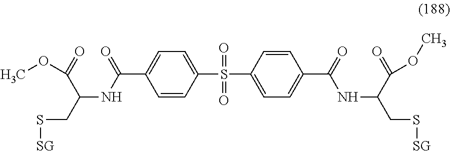

### Compound 189

Compound 187 (0.54 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 189. The purity is checked with thin layered chromatography on PEI cellulose matrix developed with 0.4M ammonium bicarbonate solution.

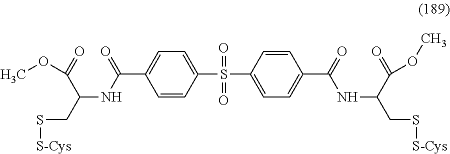

### Compound 190-198

Starting Material: Biphenyl-4,4′-dicarboxylic acid. Aldrich Catalog number: 225266. M.W: 242.23

### Compound 190

Biphenyl-4,4′-dicarboxylic acid (2.42 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 190.

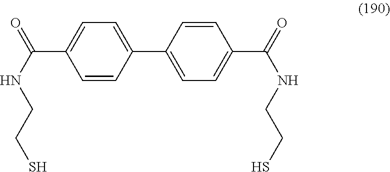 [SMILES: O=C(NCCS)c1ccc(-c2ccc(C(=O)NCCS)cc2)cc1]

### Compound 191

Compound 190 (0.360 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 191. The purity is checked with thin layered chromatography on PEI cellulose matrix developed with 0.4M ammonium bicarbonate solution.

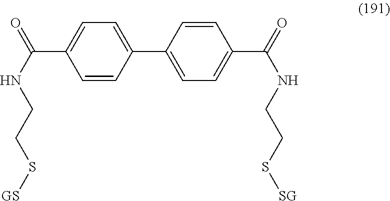

### Compound 192

Compound 190 (0.362 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 192. The purity is checked with thin layered chromatography on PEI cellulose matrix developed with 0.4M ammonium bicarbonate solution.

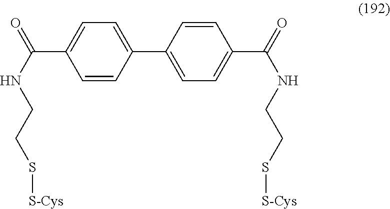

### Compound 193

Biphenyl-4,4′-dicarboxylic acid (2.42 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 193.

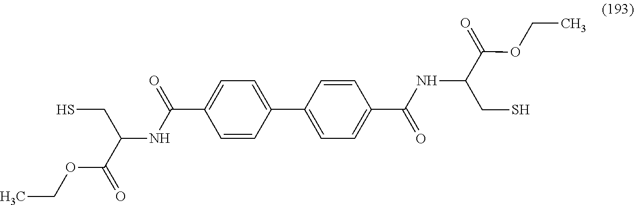 [SMILES: CCOC(=O)C(CS)NC(=O)c1ccc(-c2ccc(C(=O)NC(CS)C(=O)OCC)cc2)cc1]

### Compound 194

Compound 193 (0.504 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 194. The purity is checked with thin layered chromatography on PEI cellulose matrix developed with 0.4M ammonium bicarbonate solution.

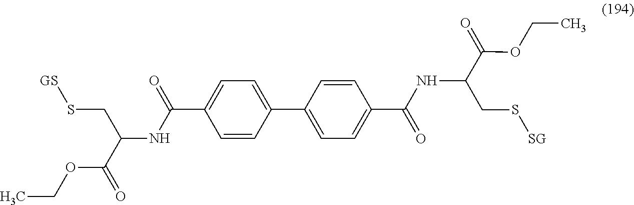

### Compound 195

Compound 193 (0.504 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 195 The purity is checked with thin layered chromatography on PEI cellulose matrix developed with 0.4M ammonium bicarbonate solution.

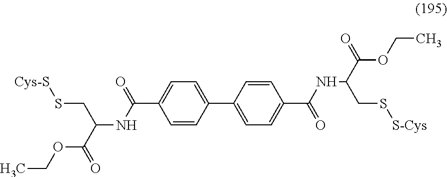

### Compound 196

Biphenyl-4,4′-dicarboxylic acid (2.42 g, 10 mmol) is taken in a 100 ml round bottomed flask. Methylene chloride (50 ml) is added to the flask. Oxalyl chloride (2.52 gm, 20 mmol) is added to the flask. 2 drops of DMF is added as a catalyst. The system is flushed with nitrogen and refluxed for 24 hours. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 196.

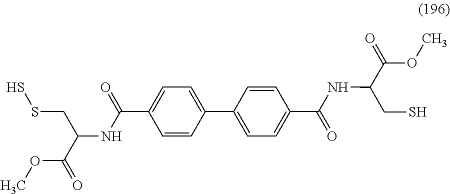

### Compound 197

Compound 196 (0.476 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 197. The purity is checked with thin layered chromatography on PEI cellulose matrix developed with 0.4M ammonium bicarbonate solution.

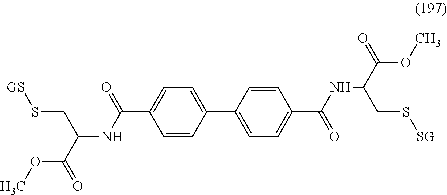

### Compound 198

Compound 196 (0.476 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 198. The purity is checked with thin layered chromatography on PEI cellulose matrix developed with 0.4M ammonium bicarbonate solution.

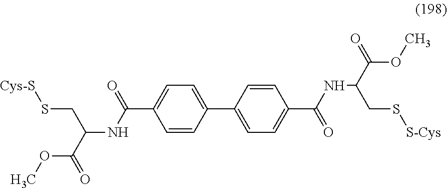

Compounds 199-207

Starting Material: 2,2′-imidobenzoic acid. Aldrich Catalog number: 308935. M.W: 257.24

### Compound 199

2,2′-imidobenzoic acid (2.57 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 199.

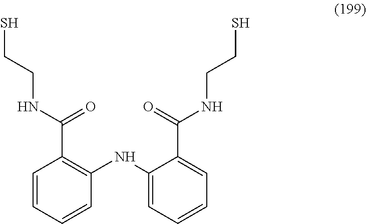 [SMILES: O=C(NCCS)c1ccccc1Nc1ccccc1C(=O)NCCS]

### Compound 200

Compound 199 (0.375 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 200. The purity is checked with thin layered chromatography on PEI cellulose matrix developed with 0.4M ammonium bicarbonate solution.

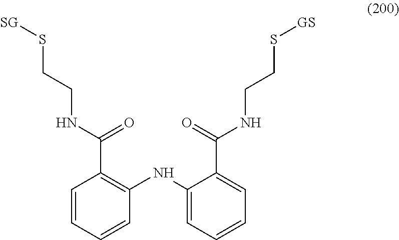

### Compound 201

Compound 199 (0.375 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 201. The purity is checked with thin layered chromatography on PEI cellulose matrix developed with 0.4M ammonium bicarbonate solution.

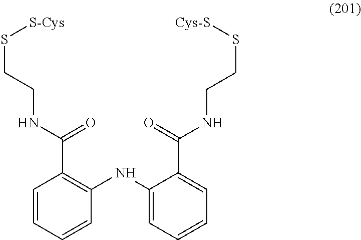

### Compound 202

2,2′-imidobenzoic acid (2.57 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 202.

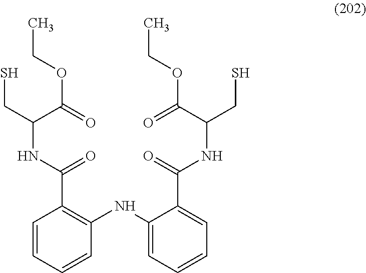 [SMILES: CCOC(=O)C(CS)NC(=O)c1ccccc1Nc1ccccc1C(=O)NC(CS)C(=O)OCC]

### Compound 203

Compound 202 (0.519 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 203. The purity is checked with thin layered chromatography on PEI cellulose matrix developed with 0.4M ammonium bicarbonate solution.

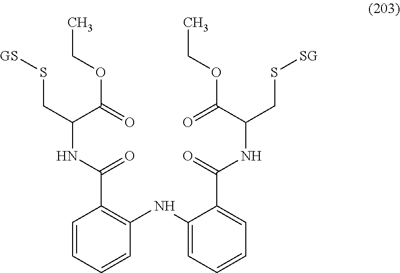

### Compound 204

Compound 202 (0.519 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 204. The purity is

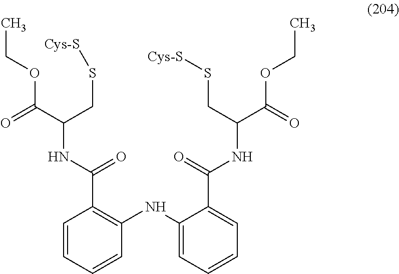

### Compound 205

2,2′-imidobenzoic acid (2.57 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 205.

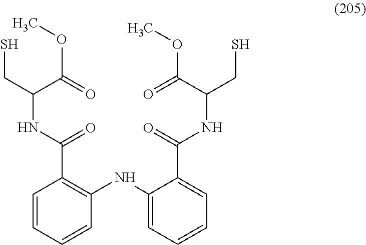 [SMILES: COC(=O)C(CS)NC(=O)c1ccccc1Nc1ccccc1C(=O)NC(CS)C(=O)OC]

### Compound 206

Compound 205 (0.491 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 206. The purity is checked with thin layered chromatography on PEI cellulose matrix developed with 0.4M ammonium bicarbonate solution.

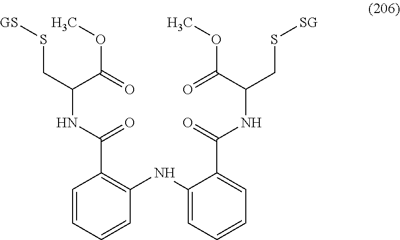

### Compound 207

Compound 205 (0.491 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 207. The purity is checked with thin layered chromatography on PEI cellulose matrix developed with 0.4M ammonium bicarbonate solution.

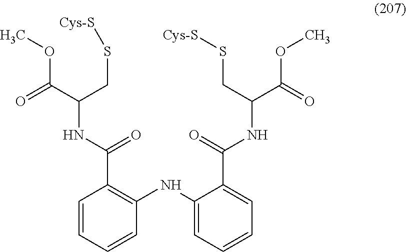

Compounds 208-216

Starting Material: 2,2′-bipyridine-5,5′-dicarboxylic acid. Aldrich Catalog number: 517763. M.W: 244.20

### Compound 208

2,2′-bipyridine-5,5′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 208.

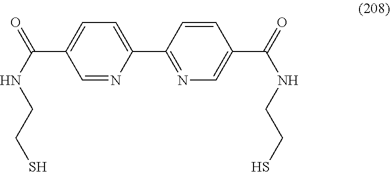 [SMILES: O=C(NCCS)c1ccc(-c2ccc(C(=O)NCCS)cn2)nc1]

### Compound 209

Compound 208 (0.362 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 209. The purity is checked with thin layered chromatography on PEI cellulose matrix developed with 0.4M ammonium bicarbonate solution.

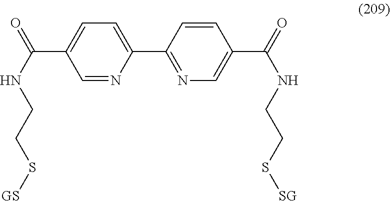

### Compound 210

Compound 208 (0.362 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 210. The purity is checked with thin layered chromatography on PEI cellulose matrix developed with 0.4M ammonium bicarbonate solution.

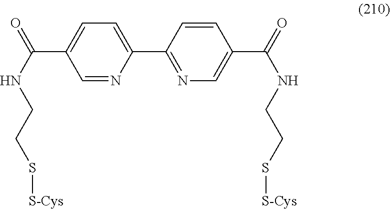

### Compound 211

2,2′-bipyridine-5,5′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 211.

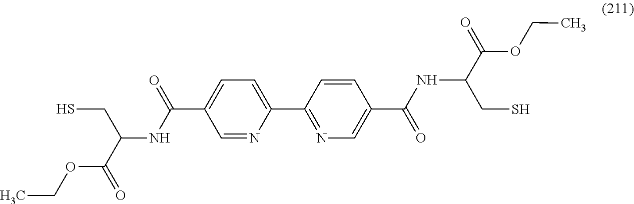 [SMILES: CCOC(=O)C(CS)NC(=O)c1ccc(-c2ccc(C(=O)NC(CS)C(=O)OCC)cn2)nc1]

### Compound 212

Compound 211 (0.506 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 212. The purity is checked with thin layered chromatography on PEI cellulose matrix developed with 0.4M ammonium bicarbonate solution.

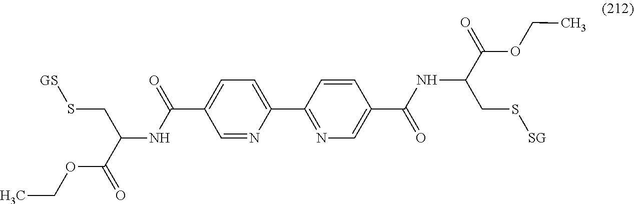

### Compound 213

Compound 211 (0.506 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 213. The purity is checked with thin layered chromatography on PEI cellulose matrix developed with 0.4M ammonium bicarbonate solution.

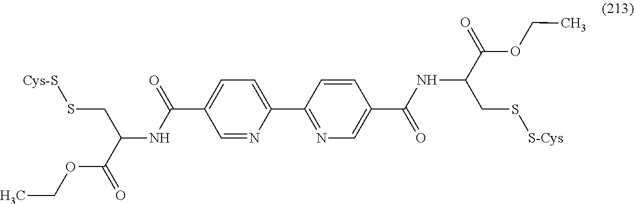

### Compound 214

2,2′bipyridine-5,5′-dicarboxylic acid (2.44 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 214.

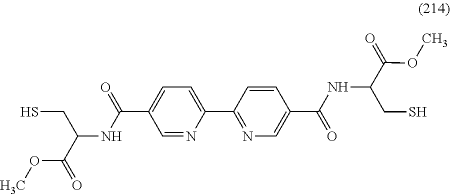

### Compound 215

Compound 214 (0.478 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 215. The purity is checked with thin layered chromatography on PEI cellulose matrix developed with 0.4M ammonium bicarbonate solution.

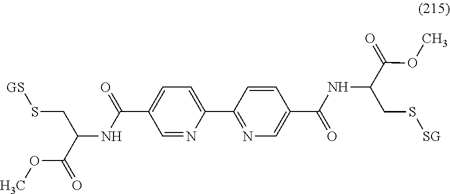

### Compound 216

Compound 214 (0.478 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 216. The purity is checked with thin layered chromatography on PEI cellulose matrix developed with 0.4M ammonium bicarbonate solution.

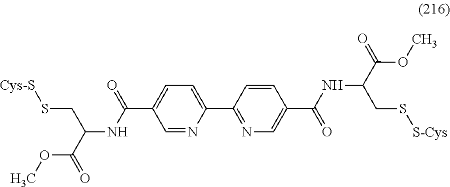

### Compounds 217-225

Starting Material: 1,1-Doxo-2,6-diphenyl thiomorpholine-3,5 dicarboxylic acid dimethyl ester. Aldrich Catalog number: S-246719. M.W: 403.457

### Compound 217

1,1-Doxo-2,6-diphenyl thiomorpholine-3,5 dicarboxylic acid dimethyl ester (4.03 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

Cysteamine hydrochloride (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the cysteamine hydrochloride has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 217.

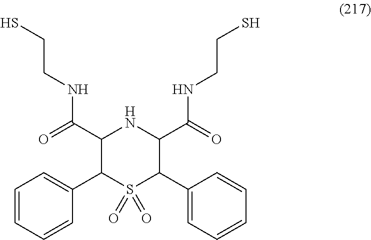 [SMILES: O=C(NCCS)C1NC(C(=O)NCCS)C(c2ccccc2)S(=O)(=O)C1c1ccccc1]

### Compound 218

Compound 217 (0.493 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 218. The purity is checked with thin layered chromatography on PEI cellulose matrix developed with 0.4M ammonium bicarbonate solution.

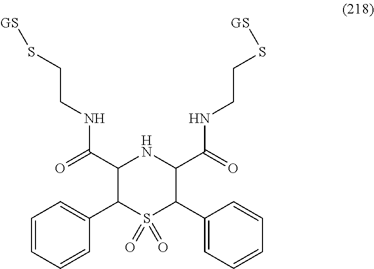

### Compound 219

Compound 217 (0.493 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 219. The purity is checked with thin layered chromatography on PEI cellulose matrix developed with 0.4M ammonium bicarbonate solution.

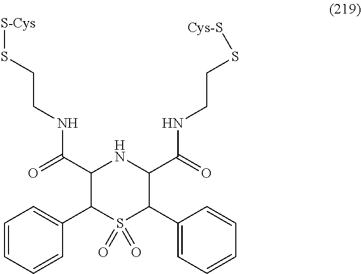

### Compound 220

1,1-Doxo-2,6-diphenyl thiomorpholine-3,5 dicarboxylic acid dimethyl ester (4.03 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride ethyl ester (2.825 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride ethyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding d.i water (15 ml). The mixture is separated on a separatory column. The organic layer is collected and washed three times with d.i water (15 ml×3). The layer is evaporated to collect the crude product. The crude product is purified on a silica column to obtain compound 220.

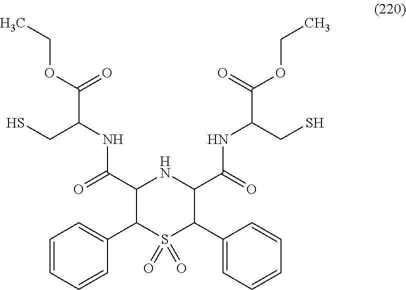 [SMILES: CCOC(=O)C(CS)NC(=O)C1NC(C(=O)NC(CS)C(=O)OCC)C(c2ccccc2)S(=O)(=O)C1c1ccccc1]

### Compound 221

Compound 220 (0.637 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 221. The purity is checked with thin layered chromatography on PEI cellulose matrix developed with 0.4M ammonium bicarbonate solution.

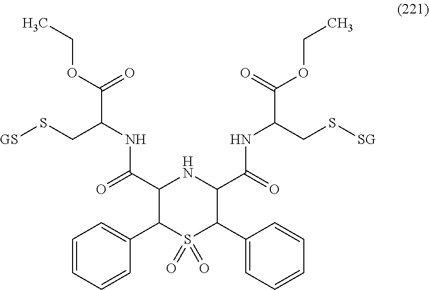

### Compound 222

Compound 220 (0.637 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 222. The purity is checked with thin layered chromatography on PEI cellulose matrix developed with 0.4M ammonium bicarbonate solution.

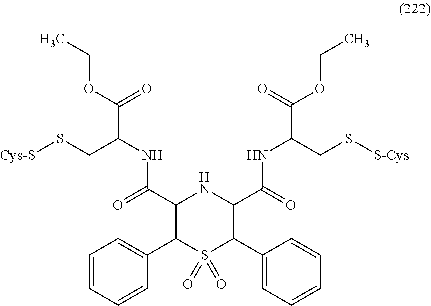

### Compound 223

1,1-Doxo-2,6-diphenyl thiomorpholine-3,5 dicarboxylic acid dimethyl ester (4.03 g, 10 mmol) is taken in a 100 ml round bottomed flask. Methylene chloride (50 ml) is added to the flask. Oxalyl chloride (2.52 gm, 20 mmol) is added to the flask. The system is flushed with nitrogen and stirred for 24 hours. The reaction mixture may be warmed or refluxed if the progress is slow. The progress is monitored using thin layer chromatography. The resulting acid chloride is used in the next step.

L-cysteine hydrochloride methyl ester (2.625 gm, 25 mmol) is dissolved in a 100 ml round bottomed flask and Methylene chloride (50 ml) is added to it and stirred. Triethylamine (3.1 gm, 30 mmol) is added to it drop by drop under constant stirring. Once all the L-cysteine hydrochloride methyl ester has dissolved the acid chloride generated in the earlier step is added to the reaction mixture drop wise. Triethylamine (5.15 gm, 50 mmol) is added to the mixture and the stirring continued for around 12 hours. The reaction progress is monitored using thin layered chromatography. The reaction is quenched by adding water (15 ml). The mixture is separated on a separatory column. The organic layer is collected and washed three times with d.i water (15 ml×3). The layer is evaporated to collect the crude product. The crude product is purified on a silica column to obtain compound 223.

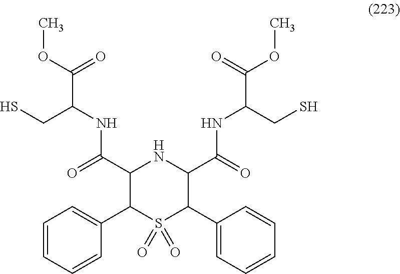 [SMILES: COC(=O)C(CS)NC(=O)C1NC(C(=O)NC(CS)C(=O)OC)C(c2ccccc2)S(=O)(=O)C1c1ccccc1]

### Compound 224

Compound 223 (0.609 gm, 1 mmol) is dissolved in 10 ml of Dimethyl formamide and 5 ml of water is added to the mixture. Glutathione (0.76 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is Compound 224. The purity is checked with thin layered chromatography on PEI cellulose matrix developed with 0.4M ammonium bicarbonate solution.

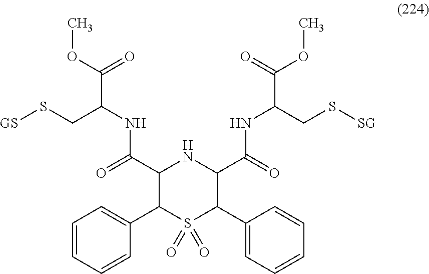

### Compound 225

Compound 223 (0.491 gm, 1 mmol) is dissolved in 10 ml of Dimethyl formamide and 5 ml of water is added to the mixture.

L-cysteine (0.50 gm) is added to the mixture and stirred till it dissolved. The reaction mixture is purged with nitrogen and stirred for 30 minutes. 1 ml of 5% Hydrogen peroxide is added to it and the reaction stirred for approximately 24 hours at room temperature with regular monitoring using thin layer chromatography. The reaction mixture loaded onto a DEAE cellulose column (2 cm×20 cm long) in the hydroxide form and washed with 200 ml of distilled water. Bound material is eluted using a 0-400 mM gradient of triethylammonium bicarbonate (TEAB) buffer with 10 ml fractions being collected. Elution of compound 2 containing product is monitored by a ultraviolet flow through device. Fractions collected containing the UV absorbance is evaporated to dryness over four co-evaporations with methanol/water to remove TEAB. The resulting material is compound 225. The purity is

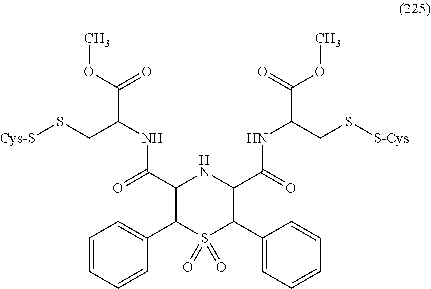

Other Compounds

In the synthesis examples for compounds 2,5, 8, 11, 14, 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221 and 224 the glutathione is replaced with one of the following compounds: alphadihydrolipoic acid, cystamine, thiolphosphate, 5′thioladenosine, L-homocysteine, co-enzyme A, 2-mercaptoethanol, dithiothreitol, iodoacetate, bromoacetate, fluoroacetate or chloroacetate.

The hydrogen peroxide causes oxidation of the two —SH groups on each compound to be oxidized to a disulfide linkage (—S—S—).

What is claimed:

1. A chemical compound comprising: 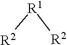
where R¹= 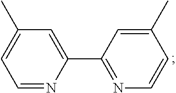
and
where R²= 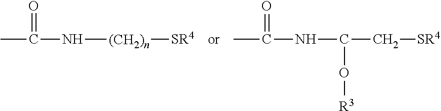
where R³=ethyl or methyl, R⁴=hydrogen, glutathione, cysteine, alphadihydrolipoic acid, cystamine, thiolphosphate, 5′thioladenosine, L-homocysteine, co-enzyme A, 2-mercaptoethanol, dithiothreitol, iodoacetate, bromoacetate, fluoroacetate or chloroacetate and n=2-4.

2. The compound of claim 1 wherein R²= 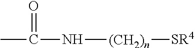
and n=2-4.

3. The compound of claim 2 wherein n=2.

4. The compound of claim 3 wherein R⁴═H.

5. The compound of claim 2 wherein n=3.

6. The compound of claim 5 wherein R⁴═H.

7. The compound of claim 2 wherein n=4.

8. The compound of claim 7 wherein R⁴═H.

9. The compound of claim 1 wherein R²= 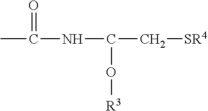
and n=2-4.

10. The compound of claim 9 wherein n=2.

11. The compound of claim 10 wherein R⁴═H.

12. The compound of claim 9 wherein n=3.

13. The compound of claim 12 wherein R⁴═H.

14. The compound of claim 9 wherein n=4.

15. The compound of claim 14 wherein R⁴═H.

16. The compound of claim 1 wherein
R¹= 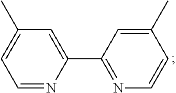
R²= 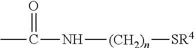
R⁴═H; and
n=2.
